(19) United States
(12) Patent Application Publication
(10) Pub. No.: US 2022/0022457 A1
(43) Pub. Date: Jan. 27, 2022

(54) Microbicide Ammonium-Imidazolium Oligomers and Their Anti-Fungal Compositions

(71) Applicant: AGENCY FOR SCIENCE, TECHNOLOGY AND RESEARCH, Singapore (SG)

(72) Inventors: Yugen Zhang, Singapore (SG); Yuan Yuan, Singapore (SG)

(21) Appl. No.: 17/276,866

(22) PCT Filed: Sep. 20, 2019
(86) PCT No.: PCT/SG2019/050476

§ 371 Date: Mar. 17, 2021

(30) Foreign Application Priority Data
Sep. 20, 2018 (SG) 10201808210R

(51) Int. Cl. A01N 43/90; A01N 43/653; C07D 519/00

(52) U.S. Cl. CPC A01N 43/90; C07D 519/00; A01N 43/653

(57) ABSTRACT

The present disclosure relates to compositions comprising an oligomer of Formula (I):

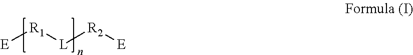

having imidazolium and diammonium substituents; and an anti-fungal compound comprising at least one triazole group. The composition as described herein may be used as an anti-fungal composition for therapeutic and non-therapeutic applications.

### CROSS-REFERENCE TO RELATED APPLICATION

This application claims priority to Singapore application number 10201808210R filed on 20 Sep. 2018, the disclosure of which is hereby incorporated by reference.

### TECHNICAL FIELD

The present invention relates to imidazolium and diammonium-based oligomers, specifically oligomers which may demonstrate antimicrobial activity. Such oligomers may be used in an antimicrobial composition with other known antimicrobial compounds for therapeutic and non-therapeutic purposes.

### BACKGROUND

Antimicrobial resistance is one of the major global healthcare threats facing our society today. Its development may be attributed to the overuse of antibiotics which are applied in various fields, including agriculture and medicine. The emergence of resistance against antibiotics has spurred the search for new antimicrobial compounds with new modes of action.

The development of compounds with anti-fungal activity is of particular interest as the number of effective anti-fungal drugs available in the market remains rather limited. To date, azoles remain one of the main classes of anti-fungal compounds. Like many other antimicrobial drugs, its extensive overuse has led to the onset of resistance.

Anti-microbial peptides (AMPs) have emerged as a class of new therapeutic antimicrobials which show good potential due to its potent and broad spectrum of activity against various microorganisms including bacteria, fungi and virus. The net positive charge of the AMPs may allow the AMPs to induce cell death through alternative modes of action. However, the high cost of its manufacture, possible proteolytic degradation and in vivo toxicity have severely limited the development of AMPs as antibiotics.

Recent studies have found that imidazolium and diammonium synthetic polymers are potential mimics of AMPs. Several of these synthetic polymers have displayed promising in vivo and in vitro activity. Despite this, there remains a concern regarding the potential clinical application of these synthetic polymers, particularly with regard to the heterogeneity and toxicity related to these high molecular weight compounds.

Considering the limited development of new anti-fungal compounds, researchers and clinicians alike are now looking into combination therapy as an alternative for the treatment of fungal infections such as candidiasis, which is caused by fungal pathogens such as Candida albicans. Azole anti-fungal compounds have most commonly been used in combination with antibiotics and other anti-fungals. However, most in vitro studies have demonstrated mixed results of antagonism and indifference. These problems underlie the need for novel antifungal agents or improved therapeutic strategies.

It is an object of the present invention provide novel imidazolium or diammonium based oligomers or polymers which may demonstrate anti-fungal activity. It is further an object of the present invention to provide alternative therapeutic strategies which may be used for the treatment of fungal infections. In particular, it is desirable to provide therapeutic alternatives which do not induce resistance.

### SUMMARY OF INVENTION

In one aspect of the present disclosure, there is provided a composition comprising: (a) an oligomer of Formula (I)

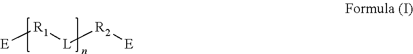

wherein R₁ is in each instance, same or different, and is independently selected from the group consisting of:

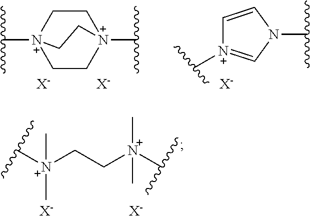

R₂ is independently selected from the group consisting of

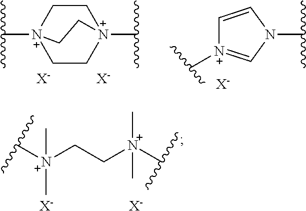

wherein X, in each instance, is same or different, and is a halogen;

L is, in each instance, independently selected from the group consisting of: optionally substituted alkyl, optionally substituted alkenyl, optionally substituted alkynyl, optionally substituted aryl, optionally substituted arylalkyl, optionally substituted arylalkenyl, optionally substituted arylalkynyl, optionally substituted alkylaryl, optionally substituted alkenylaryl, and optionally substituted alkynylaryl;

E consists of between 2 to 20 carbon atoms and is independently selected from the group consisting of: optionally substituted alkyl, optionally substituted aryl, optionally substituted arylalkyl and optionally substituted alkylaryl;

n is an integer of between 1 to 10; and

(b) an anti-fungal agent comprising at least one triazole group.

Advantageously, it has been found that the compositions disclosed herein are particularly effective for the treatment of microbial infections and for the inhibition of the growth of microbes. Specifically, it has been observed that the antimicrobial effects of the combination of the oligomer component and the anti-fungal component are greater than the additive effects of each component alone. This may be attributed to the additive effect of the fungicidal properties of the oligomer and the fungistatic properties of the triazole anti-fungal agent.

In another aspect, the present disclosure relates to pharmaceutical and non-pharmaceutical uses of the compositions defined herein. In one aspect, the present disclosure provides the use of the composition described herein in the preparation of a medicament for the treatment of microbial infections.

Surprisingly, the present composition is able to prevent the development of resistance when used as an anti-microbial agent. In particular, it was surprisingly found that the effective therapeutic concentration which may be used for the treatment of anti-microbial infections does not decrease even after extensive use throughout the lifetime of the microorganism. The composition was found to be effective even as passage number of the microorganism increases.

### BRIEF DESCRIPTION OF DRAWINGS

FIG. 1 is a plot of the number of surviving Candida albicans colonies upon treatment with selected oligomers at a concentration of 62 μg/ml. C. albicans grown in Yeast Mold broth was used as control while C. albicans treated with fluconazole (flu) was used for comparison. The data are expressed as the mean±standard deviation of triplicates.

FIG. 2a is a plot of the percentage growth of Staphylococcus aureus after 24 hours of incubation with varied concentrations of norfloxacin, IDPBX8 and a mixture of norfloxacin and IDPBX8 in a 1:1 weight ratio.

FIG. 2b is a plot of the percentage growth of Candida albicans after 24 hours of incubation with varied concentrations of fluconazole, IDPBX8 and a mixture of fluconazole and IDPBX8 in a 1:1 weight ratio. The percentage growth was calculated based on the absorbance of the cell culture at 600 nm, measured by a plate reader.

FIG. 2c is a plot of the number of colony forming units of Candida albicans after 24 hours of incubation with varied concentrations of fluconazole, IDPBX8 and a mixture of fluconazole and IDPBX8 in a 1:1 weight ratio. The concentration of Candida albicans at 0 hours is 3.8×10⁶ CFU/ml as indicated in the figure. The data are expressed as the mean±standard deviation of triplicates.

FIG. 3a is a plot of the number of colony forming units (CFU) of Candida albicans at different time intervals after incubation with 1 μg/ml of IDPBX8; or 0.5 μg/ml fluconazole alone; or a mixture of 1 μg/ml of IDPBX8 and 0.5 μg/ml of fluconazole (Combination-1). The data are expressed as the mean±standard deviation of triplicates.

FIG. 3b is a plot of the number of colony forming units of Candida albicans at different time intervals after incubation with 2 μg/ml of the IDPBX8 oligomer or 0.25 μg/ml of fluconazole, or a mixture of 2 μg/ml of the IDPBX8 oligomer and 0.25 μg/ml of fluconazole (Combination-2). The data are expressed as the mean±standard deviation of triplicates.

FIG. 4 is a plot of the normalized MIC values against the passage number of a Candida albicans culture. This illustrates the acquisition of resistance by the Candida albicans culture which is grown in the presence of ¼ MIC levels of IDPBX8, fluconazole or fluconazole-IDPBX8 combinations. Combination-1 refers to a mixture of 1 μg/ml IDPBX8 oligomer and 0.5 μg/ml fluconazole, while combination-2 refers to a mixture of 2 μg/ml IDPBX8 oligomer and 0.25 μg/ml fluconazole.

### DEFINITIONS

The following words and terms used herein shall have the meaning indicated:

In the definitions of a number of substituents below it is stated that “the group may be a terminal group or a bridging group”. This is intended to signify that the use of the term is intended to encompass the situation where the group is a linker between two other portions of the molecule as well as where it is a terminal moiety. Using the term alkyl as an example, some publications would use the term “alkylene” for a bridging group and hence in these other publications there is a distinction between the terms “alkyl” (terminal group) and “alkylene” (bridging group). In the present application no such distinction is made and most groups may be either a bridging group or a terminal group.

The term “alkyl” as a group or part of a group refers to a straight or branched aliphatic hydrocarbon group having but not limited to, from 1 to 16 carbon atoms, e.g., 1, 2, 3, 4, 5, 6, 7, 8, 9, 10, 11, 12, 13, 14, 15 or 16 carbon atoms, preferably a C₁-C₁₆ alkyl, C₁-C₁₂ alkyl, more preferably a C₁-C₁₀ alkyl, most preferably C₁-C₆ alkyl, unless otherwise noted. Examples of suitable straight and branched alkyl substituents include but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 5-methylheptyl, 1-methylheptyl, octyl, nonyl, decyl, undecyl, 2,2,3-trimethyl-undecyl, dodecyl, 2,2-dimethyl-dodecyl, tridecyl, 2-methyl-tridecyl, 2-methyltridecyl, tetradecyl, 2-methyl-tetradecyl, pentadecyl, 2-methyl-pentadecyl, hexadecyl, 2-methyl-hexadecyl and the like. The group may be a terminal group or a bridging group. The alkyl may be optionally substituted with one or more groups as defined under the term “optionally substituted” below.

The term “aryl” as a group or part of a group to be interpreted broadly denotes (i) an optionally substituted monocyclic, or fused polycyclic, aromatic carbocycle (ring structure having ring atoms that are all carbon) preferably having from 5 to 12 atoms per ring, wherein the optionally substitution can be di-substitution, or tri-substitution. Examples of aryl groups include phenyl, naphthyl, and the like; (ii) an optionally substituted partially saturated bicyclic aromatic carbocyclic moiety in which a phenyl and a C₅-C₇ cycloalkyl or C₅-C₇ cycloalkenyl group are fused together to form a cyclic structure, such as tetrahydronaphthyl, indenyl or indanyl. The group may be a terminal group or a bridging group. Typically an aryl group is a C₆-C₂₀ aryl group. The aryl may be optionally substituted with one or more groups as defined under the term “optionally substituted” below.

The term “arene” as used herein refers to hydrocarbons with sigma bonds and delocalized pi electrons between carbon atoms forming a circle. The arene may also refer to an aromatic hydrocarbon. The arene may be monocyclic or polycyclic. The arene may have but not limited to, at least 6 carbon atoms, 6 to 20 carbon atoms, or 6 to 12 carbon atoms. Examples of arene include but not limited to, benzene, methylbenzene, ethylbenzene, xylene, and diethylbenzene. The arene may be optionally substituted with one or more groups as defined under the term “optionally substituted” below.

The term “alkyloxy” or “alkoxy” refers to an alkyl-O— group to be interpreted broadly in which alkyl is as defined herein. The alkyloxy is a C₁-C₁₆ alkyloxy, C₁-C₁₂ alkyloxy, more preferably a C₁-C₁₀ alkyloxy, most preferably C₁-C₆ alkyloxy. Examples include, but are not limited to, methoxy, ethoxy and propoxy. The group may be a terminal group or a bridging group. The term alkyloxy may be used interchangeably with the term “alkoxy”. The alkyloxy or alkoxy may be optionally substituted with one or more groups as defined under the term “optionally substituted” below.

The term “alkenyl” as a group or part of a group denotes an aliphatic hydrocarbon group containing at least one carbon-carbon double bond and which may be straight or branched having but not limited to, at least 2 carbon atoms, 2-20 carbon atoms, 2-10 carbon atoms, 2-6 carbon atoms, or any number of carbons falling within these ranges, in the normal chain. The group may contain a plurality of double bonds in the normal chain and the orientation about each is independently E, Z, cis or trans where applicable. Exemplary alkenyl groups include, but are not limited to, ethenyl, propenyl, butenyl, pentenyl, hexenyl, heptenyl, octenyl and nonenyl. The group may be a terminal group or a bridging group. The alkenyl may be optionally substituted with one or more groups as defined under the term “optionally substituted” below.

The term “alkynyl” as used herein includes within its meaning unsaturated aliphatic hydrocarbon groups having but not limited to, at least 2 carbon atoms or 2 to 20 carbon atoms, and having at least one triple bond anywhere in the carbon chain. Examples of alkynyl groups include but are not limited to ethynyl, 1-propynyl, 1-butynyl, 2-butynyl, 1-methyl-2-butynyl, 3-methyl-1-butynyl, 1-pentynyl, 1-hexynyl, methylpentynyl, 1-heptynyl, 2-heptynyl, 1-octynyl, 2-octynyl, 1-nonyl, 1-decynyl, and the like. The group may be a terminal group or a bridging group. The alkynyl may be optionally substituted with one or more groups as defined under the term “optionally substituted” below.

The term “halo” or “halogen” as used herein refers to fluorine, chlorine, bromine and iodine while the term “halide” as used herein refers to fluoride, chloride, bromide and iodide.

The term “alcohol” as used herein refers to compounds in which the hydroxyl functional group (—OH) is bound to a carbon. The alcohol may have but is not limited to, at least 1 carbon atom, 1 to 20 carbon atoms, 1 to 12 carbon atoms, 1 to 6 carbon atoms, 2 to 6 carbon atoms, or 2 to 4 carbon atoms. Examples of alcohol include but are not limited to, methanol, ethanol, propan-1-ol, propan-2-ol, 2-methylpropan-1-ol, 2-methylpropan-2-ol, butan-1-ol and butan-2-ol. The alcohol may be optionally substituted with one or more groups as defined under the term “optionally substituted” below.

The term “optionally substituted”, as used in the context of the present disclosure, means the group to which this term refers may be unsubstituted, or may be substituted with one or more groups independently selected from alkyl, alkenyl, alkynyl, thioalkyl, cycloalkyl, cycloalkylalkyl, cycloalkenyl, cycloalkylalkenyl, heterocycloalkyl, cycloalkylheteroalkyl, cycloalkyloxy, cycloalkenyloxy, haloalkyl, haloalkenyl, haloalkynyl, alkynyloxy, heteroalkyl, heteroalkyloxy, alkoxy, alkenyloxy, haloalkoxy, haloalkenyloxy, alkyloxy, alkyloxyalkyl, alkyloxyaryl, alkyloxycycloalkyl, alkyloxyheteroaryl, alkyloxyheterocycloalkyl, alkenoyl, alkynoyl, heterocyclic, heterocycloalkenyl, heterocycloalkyl, heterocycloalkylalkyl, heterocycloalkylalkenyl, heterocycloalkylheteroalkyl, heterocycloalkyloxy, heterocycloalkenyloxy, heterocycloxy, haloheterocycloalkyl, aryl, heteroaryl, heteroarylalkyl, heteroarylalkenyl, heteroarylheteroalkyl, heteroaryloxy, arylalkenyl, arylalkyl, alkylaryl, alkylheteroaryl, aryloxy.

The term “charge density” may refer to the ratio of the charge of an ionic compound to its volume. As used herein, the term may refer to the ratio of the ionic charge of an oligomer or polymer to its length.

The term “amphiphilic” as used herein refers to compounds having a structure or a conformation comprising discrete hydrophilic and hydrophobic regions. These hydrophilic and hydrophobic regions may be arranged in an alternating or sequential manner.

The term “microorganism” as used herein, refers broadly to both eukaryotic and prokaryotic organisms possessing a cell membrane, including but not limited to, bacteria, yeasts, fungi, plasmids, algae and protozoa.

The term “minimum inhibitory concentration (MIC)” as used herein refers to the concentration of an antimicrobial compound or composition at which no meaningful microorganism growth was observed. The growth of microorganisms may be detected through cell counting methods, microscopy techniques, by measuring the weight of cells isolated from culture media, or by measuring the turbidity of the culture medium. The turbidity of the culture medium may be measured using a turbidimeter, or by spectroscopic means, such as by determining optical density of the medium at a specific wavelength.

The term “MIC₅₀” as used herein refers to the concentration of an antimicrobial agent which is able to reduce the growth of the microorganism by 50%.

The term “fractional inhibitory concentration (FIC)” as used herein refers to an index intended to estimate the interaction between two or more compounds intended to be used in combination. The index may be determined by normalizing the MIC of each compound when used in a combination with the MIC of the compound when use as a sole therapeutic agent. The FIC of a combination of two therapeutic agents may be determined according to the formula below. Combinations which may demonstrate synergistic effects may have an FIC index of less than 0.5, while combinations which result in indifference may have an FIC index of more than 0.5

$$Fractional Inhibitory Concentration ( FIC ) = \frac{{MIC}_{Compound A in composition}}{{MIC}_{Compound A alone}} + \frac{{MIC}_{Compound B in composition}}{{MIC}_{Compound B alone}}$$

The term ‘antagonist’ as used herein refers to compounds which interfere or block the activity of another therapeutic compound. Compounds which exhibit antagonist activity are able to reduce the effectiveness of other therapeutic compounds which it may be administered with.

The term ‘synergistic’ as used herein refers to the interaction or cooperation of two or more compounds which, together, produces a combined effect which is greater than the sum of their separate effects. Compositions which exhibit synergism may be able to reduce the population of microorganisms more effectively than the individual components of the composition. More specifically, compositions which exhibit synergism may exhibit a lower MIC or MIC50 value as compared to their individual components.

The term ‘monomer’ as used herein refers to a compound which may react chemically with other molecules which may or may not be of the same type to form a larger molecule.

The term ‘oligomer’ as used herein refers to compounds which comprise repeating units of at least one monomer. Oligomers may contain less than 20 repeating units of a monomer. Examples of oligomers include dimers, trimers and tetramers which contain 2, 3 and 4 units of one or more monomers, respectively.

The term “polymer” as used herein refers to compounds which comprise multiple repeating units of a monomer. Polymers may be longer than oligomers and may comprise an infinite number of repeating units of a monomer. Polymers have long chains of repeating units and have high molecular weight.

The term “hemolysis” as used herein refers to the rupturing (lysis) of red blood cells and the release of their contents (cytoplasm) into surrounding fluid (e.g. blood plasma). Hemolysis may occur inside or outside the body.

The term “ex vivo” as used herein refers to experimentation or measurements done in or on tissue from an organism in an external environment with minimal alteration of natural conditions.

The term “fungistatic” as used herein refers to the ability of the compound or composition to inhibit and halt the growth of a fungus. Compounds which are fungistatic in nature may only be able to inhibit or slow down the cell division of the fungi without killing the fungus. Consequently, fungi which are treated with fungistatic compounds may grow at a reduced rate as compared to cultures which are not treated with any compounds.

The term “fungicidal” as used herein refers to the ability of a compound or composition to induce death of fungal cells or their spores. Compounds which are fungicidal may be able to inhibit cellular process or attack particular organelles in a cell to induce death of the fungus. As such, fungicidal compounds may be able to reduce the population of viable fungal colonies.

It is to be understood that included in the family of compounds of Formula (I) are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art.

Some of the compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

The term “therapeutically effective amount” or “effective amount” as used herein refers to an amount sufficient to effect beneficial or desired clinical results. An effective amount can be administered in one or more administrations. An effective amount is typically sufficient to palliate, ameliorate, stabilize, reverse, slow or delay the progression of the diseased state.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DESCRIPTION OF THE EMBODIMENTS

In one aspect of the present disclosure, there is provided a composition comprising an oligomer of formula (I) and an anti-fungal agent comprising at least one triazole group. The composition as disclosed herein may be used for the treatment of microbial infections. The microbial infections may be a bacterial infection or a fungal infection.

Non-limiting examples of the oligomer of formula (I) are described below. The structure of the oligomer of Formula (I) is provided:

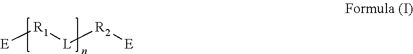

The oligomer of Formula (I) may comprise at least one positively charged unit, or at least two positively charged units, R₁ and R₂. In embodiments, the number of positively charged units in the oligomer of Formula (I) is at least 1, or between 1 to 10, or between 2 to 10, or between 2 to 9, or between 2 to 8, or between 2 to 7, or preferably between 2 to 6. In embodiments, the number of positively charged units in the oligomer is 4.

Advantageously, oligomers which comprise 2 to 6 charged units demonstrated better anti-microbial activity. The higher number of charged units may facilitate interaction of the oligomer with the charged surface of the membrane of the microbial cell. Such improved interactions may lead to improved antimicrobial activity of the oligomer against bacteria and fungi.

The positively charged R₁ and R₂ units may, in each instance, be the same or different. R₁ and R₂ may independently be a positively charged alkyl diamine group or a positively charged N-heterocyclic group. The N-heterocycle may be a 5- or 6-membered N-heterocycle. The N-heterocycle may be an aromatic N-heterocycle or a bicyclic N-heterocycle.

R₁ and R₂ may, in each instance, be independently selected from the group consisting of imidazolium, positively charged DABCO ([DABCO]²⁺) and positively charged TMEDA ([TMEDA]²⁺). The structure of imidazole, DABCO and TMEDA are shown below:

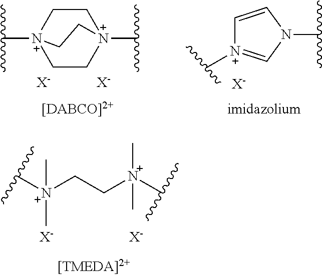

In embodiments, R₁ or R₂ may preferably be a [DABCO]2+ or imidazolium group.

R₁, in each instance, may be a positively charged alkyl diamine or a positively charged N-heterocycle. The N-heterocycle may be a 5- or 6-membered N-heterocycle. The N-heterocycle may be an aromatic N-heterocycle. R₁, in each instance, may be the same or different and may be independently selected from the group consisting of [DABCO]²⁺, imidazolium and [TMEDA]²⁺. R₁ may preferably be [DABCO]²⁺ or imidazolium groups.

R₂, in each instance, may be a positively charged alkyl diamine or a positively charged N-heterocycle. The N-heterocycle may be a 5- or 6-membered N-heterocycle. The N-heterocycle may be an aromatic N-heterocycle. R₂, in each instance, may be independently selected from the group consisting of [DABCO]²⁺, imidazolium and [TMEDA]²⁺. In embodiments, R₂ may be imidazolium.

In some embodiments, the oligomer of formula (I) comprises at least one R₁ group which is a [DABCO]²⁺ unit. In other embodiments, the oligomer of formula (I) comprises at least one [DABCO]²⁺ unit and at least one imidazolium group.

Advantageously, oligomers wherein at least one R₁ is a [DABCO]²⁺ or imidazolium group may possess improved antifungal activity as compared to oligomers which comprise long, straight chain diammonium groups. Specifically, oligomers which comprise [DABCO]²⁺ and imidazolium units may exhibit a lower MIC value against fungi as compared to oligomers which comprise long, straight chain alkyl diammonium groups. The positively charged N-heterocyclic units may lead to the formation of an oligomer of shorter chain length. This may increase the charge density of the oligomer, which may be able to induce death of fungal cells more effectively.

The positively charged R₁ and R₂ units may comprise an anion X. Anion X may be a singly charged monoatomic or polyatomic anion, preferably a monoatomic anion. Anion X in each instance, may be the same or different, and may be a halogen. In embodiments, anion X is preferably a halide such as fluoride, chloride, bromide or iodide, preferably chloride or bromide. In preferred embodiments, X may be a bromide anion.

The oligomer of Formula (I) may comprise a linker L, which is bonded to positively charged unit R₁. The linker L may, in each instance, be independently selected from the group consisting of: optionally substituted alkyl, optionally substituted alkenyl, optionally substituted alkynyl, optionally substituted aryl, optionally substituted arylalkyl, optionally substituted arylalkenyl, optionally substituted arylalkynyl, optionally substituted alkylaryl, optionally substituted alkenylaryl, and optionally substituted alkynylaryl. In embodiments, L may be an optionally substituted alkenyl, optionally substituted aryl, optionally substituted arylalkenyl or optionally substituted alkylaryl group, preferably an optionally substituted alkylaryl group.

Linker L may, in each instance, be an optionally substituted alkenyl, optionally substituted aryl, optionally substituted arylalkyl or optionally substituted alkylaryl linker comprising 2 to 20 carbon atoms, or 2 to 18 carbon atoms, or 2 to 16 carbon atoms, or 2 to 14 carbon atoms, or 2 to 12 carbon atoms, or 2 to 10 carbon atoms, or 4 to 10 carbon atoms, or preferably 6 to 10 carbon atoms. In some embodiments, L may comprise 6 carbon atoms. In other embodiments, L comprises 8 carbon atoms.

Linker L may, in each instance, be an aryl group comprising two alkyl substituents, or an alkenyl group comprising 2 alkyl substituents. L may preferably be independently selected from the group consisting of p-xylenyl, o-xylenyl and trans-2-butenyl. In some embodiments, L may, in each instance, be para-xylylene or ortho-xylylene. In other embodiments, L may be trans-2-butenyl. The structure of the para-xylylene, ortho-xylylene and trans-2-butenyl linkers L are depicted below:

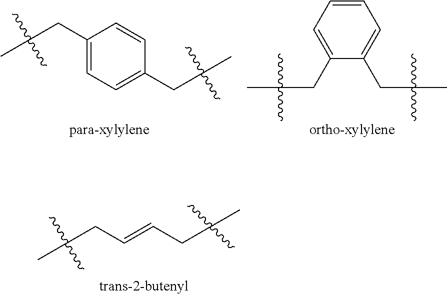 [SMILES: *Cc1ccc(C*)cc1 ; *Cc1ccccc1C* ; *C/C=C/C*]

The oligomer of Formula (I) may be capped by terminal groups E. Terminal group E may consist of 2 to 20 carbon atoms, or 2 to 18 carbon atoms, or 2 to 16 carbon atoms, or 2 to 14 carbon atoms, or 2 to 12 carbon atoms, or 4 to 12 carbon atoms, or 6 to 12 carbon atoms, preferably 6 to 10 carbon atoms. In preferred embodiments, E comprises 8 carbon atoms.

The terminal group E may be an optionally substituted aliphatic or aromatic group. E may be independently selected from the group consisting of optionally substituted alkyl, optionally substituted aryl, optionally substituted arylalkyl and optionally substituted alkylaryl. E may be an optionally substituted alkyl or optionally substituted aryl group, preferably an optionally substituted alkyl group.

In embodiments, the E terminal group may be an optionally substituted aliphatic group, preferably an aliphatic alkyl group.

Advantageously, oligomers which comprise an aliphatic E terminal group may possess improved anti-microbial activity compared to oligomers which comprise terminal aromatic groups. Without being bound by theory, it is thought that aliphatic E terminal groups may be able to achieve better interaction with the cell wall and/or hydrophobic regions of the cell membrane, allowing entry of the oligomer into the microbial cells. This may consequently allow better penetration and accumulation of the oligomer in the cell.

The aliphatic alkyl group may be a straight-chain or branched alkyl group. In some embodiments, the alkyl group may be an aliphatic alkyl group comprising 8 carbon atoms. In preferred embodiments, E is a n-octyl group.

The oligomer of Formula (I) may comprise n units of R₁-L. n may be an integer of between 1 to 20, or between 1 to 18, or between 1 to 16, or between 1 to 14, or between 1 to 12, or between 1 to 10, or between 1 to 9, or between 1 to 8, or between 1 to 7, or between 1 to 6, preferably between 1 to 5. In preferred embodiments, n is 3. Advantageously, oligomers where n is 3 demonstrate good antimicrobial activity. In particular, oligomers where n is 3 may demonstrate better efficacy against fungal infections as compared to oligomers having an n value of 1 or 2.

The oligomer of Formula (I) may be selected from the group consisting of the following oligomers:

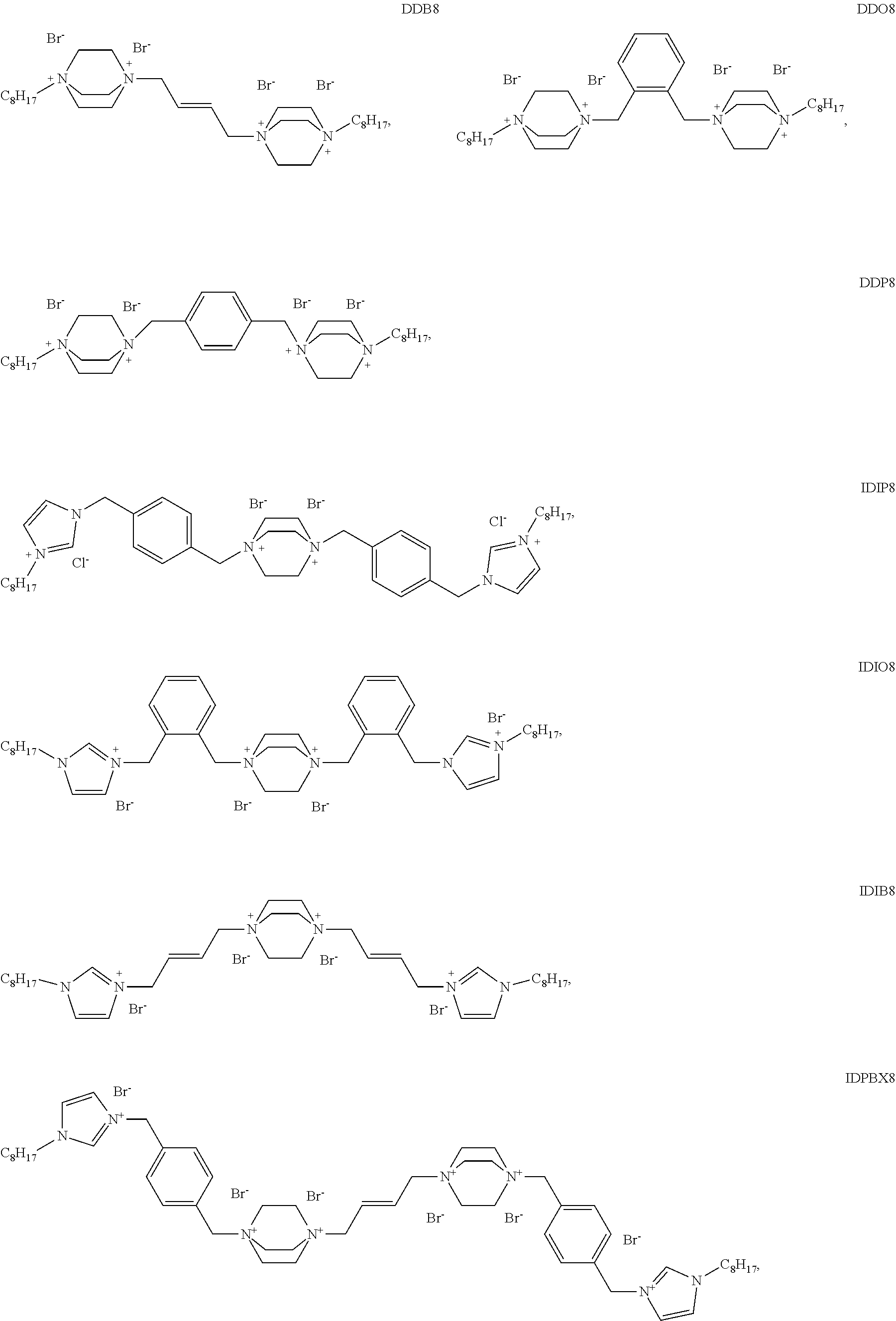

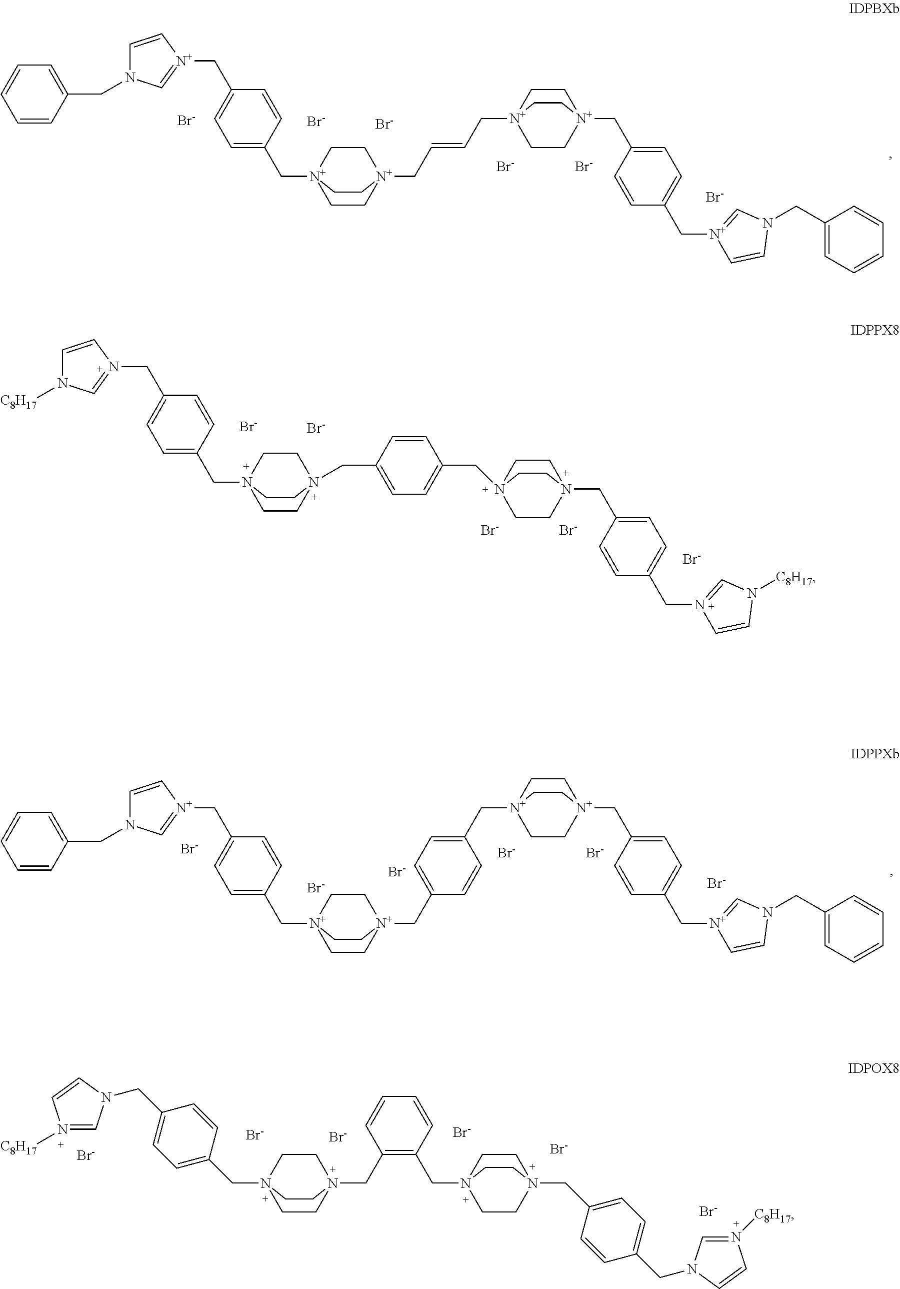

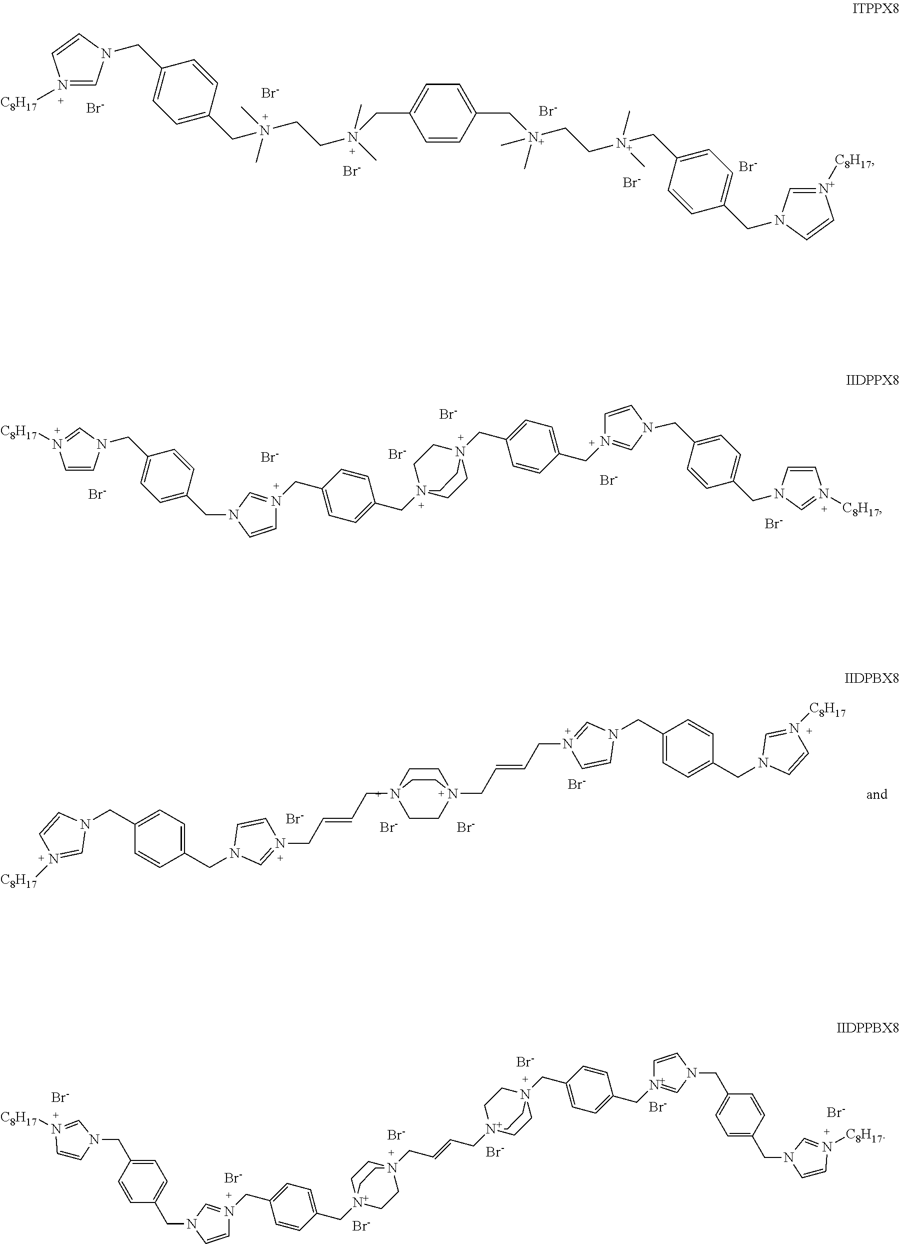

The oligomer of Formula (I) may demonstrate anti-microbial activity. The anti-microbial activity of the oligomer of Formula (I) may include anti-bacterial and anti-fungal activity. The oligomer of Formula (I) may demonstrate anti-bacterial activity against a wide spectrum of bacteria. These bacteria may include bacteria in the Staphylococcus, Pseudomonas and Escherichia family. Non-limiting examples of bacterial infections which the oligomer of Formula (I) may act against may include Staphylococcus argenteus, Staphylococcus aureus, Staphylococcus schweitzeri, Staphylococcus simiae, Staphylococcus epidermidis, Staphylococcus lugdunensis, Staphylococcus scheleiferi, Staphylococcus caprae, Pseudomonas aeruginosa, Pseudomonas oryzihabitans, Pseudomonas plecoglossicida; Escherichia hermannii and Escherichia coli. In embodiments, the oligomer of Formula (I) may demonstrate anti-bacterial activity against Staphylococcus aureus, Escherichia coli and Pseudomonas aeruginosa.

The minimal inhibitory concentration (MIC) of the oligomer of Formula (I) against bacteria may be in the range of about 1 μg/ml to about 1000 μg/ml, or about 1 ug/ml to about 800 μg/ml, or about 1 ug/ml to about 600 μg/ml, or about 1 ug/ml to about 500 μg/ml, or about 1 ug/ml to about 400 μg/ml, or about 1 ug/ml to about 300 μg/ml, or about 1 ug/ml to about 200 μg/ml, or about 1 ug/ml to about 100 μg/ml, or about 1 ug/ml to about 80 μg/ml, or about 1 ug/ml to about 60 μg/ml, or about 1 ug/ml to about 50 μg/ml, or about 1 ug/ml to about 40 μg/ml, or about 1 ug/ml to about 30 μg/ml, or about 1 ug/ml to about 20 μg/ml, or about 1 ug/ml to about 10 μg/ml. In embodiments, the oligomers of Formula (I) may inhibit the growth of bacteria at a concentration of about 2 to 8 μg/ml.

Surprisingly, short chain oligomers wherein n may be 1 or 2 may demonstrate anti-microbial activity at concentrations as low as 2-10 μg/ml. In embodiments, an oligomer of Formula (I), wherein n=1, which may comprise n-octyl E terminal groups, demonstrate anti-bacterial activity at concentrations as low as about 2-4 μg/ml against Escherichia coli.

The oligomer of Formula (I) may also demonstrate anti-fungal activity against various fungi. These fungi may include fungi and yeast from the Aspergillus, Cryptococcus and Candida family. In embodiments, the oligomer of Formula (I) may demonstrate activity against fungi from the Candida family. Non-limiting examples of fungi from the Candida family may include Candida albicans, Candida tropicalis, Candida glabrata, Candida viswanathii, Candida pseudotropicalis, Candida guillierimondii, Candida krusei, Candida lusitaniae, Candida parapsilosis and Candida stellatoidea. In embodiments, the oligomer of Formula (I) may demonstrate anti-fungal activity against Candida albicans.

Advantageously, the oligomer of Formula (I) may demonstrate anti-fungal activity against Candida albicans at concentrations as low as 8 μg/ml. Surprisingly, long chain oligomers, wherein n may be 3 to 5, which comprise flexible trans-butenyl linkers may demonstrate anti-fungal activity against Candida albicans at concentrations as low as 8 μg/ml.

Further advantageously, the oligomers as described herein demonstrate low toxicity toward mammalian cells. Hemolysis studies indicate that the concentration of the oligomer that may result in 10% hemolysis is more than 2 mg/ml, implying that even at high concentrations, the oligomer is non-toxic to mammalian cells. This is particularly desirable for oligomers which are to be used for therapeutic purposes.

The oligomers of Formula (I) may possess an amphiphilic structure and/or conformation which may facilitate the antimicrobial activity of the oligomer. In preferred embodiments, the oligomer is a fungicidal agent. Advantageously, the oligomer may be able to induce death of fungal cells and decrease the population of fungal cells.

The oligomer of Formula (I) may be used in combination with a known anti-fungal agent. The anti-fungal agent may be a fungistatic anti-fungal agent. The anti-fungal agent may comprise N-heterocyclic azole groups, preferably triazole groups. The anti-fungal agent may comprise at least one triazole group, or preferably between 1 to 5 triazole groups, or between 1 to 4 triazole groups, or between 1 to 3 triazole groups, preferably 2 triazole groups.

The triazole anti-fungal agent may be one of fluconazole, itraconazole, ketoconazole, albaconazole, ravuconazole, posaconazole or voriconazole, or combinations thereof. In embodiments, the anti-fungal agent may be fluconazole, itraconazole or voriconazole, or combinations thereof. In preferred embodiments, the anti-fungal agent of the composition may be fluconazole. The structures of fluconazole, itraconazole and voriconazole are depicted below:

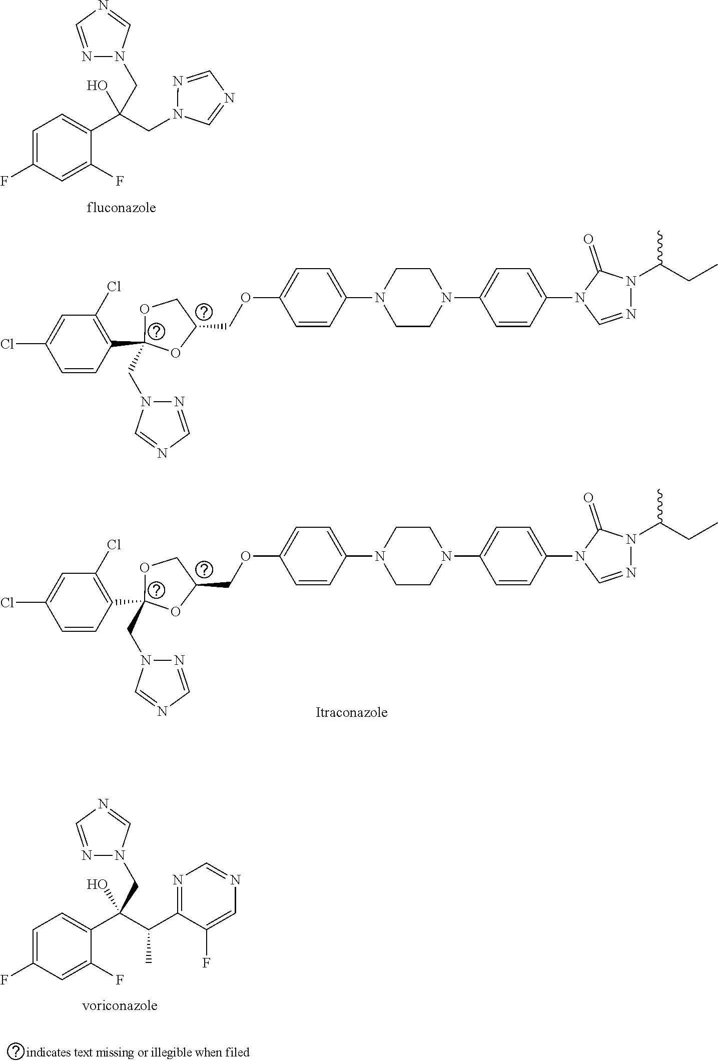

The composition as described herein, which comprises an oligomer of Formula (I) and an anti-fungal agent comprising at least one triazole ring may be used in a method for treating a microbial infection, particularly a fungal infection. The method of treatment may involve administering to a subject an effective amount of the composition described herein. The composition may be administered topically or orally, preferably by topical application on an affected area.

The composition as described herein may be used for the treatment of a microbial infection, particularly a fungal infection. The composition described herein may also be used for the preparation of a medicament for the treatment of microbial infections, particularly fungal infections. The composition or medicament may be administered at a concentration effective to treat the microbial infection. The composition or medicament may be administered topically or orally to a subject in need. The composition or medicament may preferably be administered topically on an affected area.

In another aspect, the composition as described herein may be used in a method of killing or inhibiting microbial growth ex vivo. The method may comprise the step of applying the composition described herein on an affected surface. Non-limiting examples of inanimate surfaces may include surfaces of medical devices, hospital interior surfaces, textiles, food packaging, children's toys or electrical appliances.

Microbial infections which may be treated by the composition described herein may be bacterial infections or fungal infections. Bacterial infections which may be treated using the composition described herein include infections caused by Staphylococcus, Pseudomonas and Escherichia bacteria. Non-limiting examples of bacterial infections which may be treated using the composition described herein may include Staphylococcus argenteus, Staphylococcus aureus, Staphylococcus schweitzeri, Staphylococcus simiae, Staphylococcus epidermidis, Staphylococcus lugdunensis, Staphylococcus scheleiferi, Staphylococcus caprae, Pseudomonas aeruginosa, Pseudomonas oryzihabitans, Pseudomonas plecoglossicida; Escherichia hermannii and Escherichia coli. In embodiments, the composition comprising an oligomer of Formula (I) and an anti-fungal agent may demonstrate anti-bacterial activity against Staphylococcus aureus, Escherichia coli and Pseudomonas aeruginosa.

Fungal infections which may be treated by the composition described herein include yeast infections. These yeast infections include infections caused by fungi and yeast from the Aspergillus, Cryptococcus and Candida family. In embodiments, the composition described herein may be used to treat infections caused by fungi from the Candida family. Non-limiting examples of fungi from the Candida family may include Candida albicans, Candida tropicalis, Candida glabrata, Candida viswanathii, Candida pseudotropicalis, Candida guillierimondii, Candida krusei, Candida lusitaniae, Candida parapsilosis and Candida stellatoidea. In embodiments, the composition may be used for the treatment of Candida albicans infections.

The composition used for the treatment of microbial infections may be administered at a total concentration of about 0.2 μg/ml to about 150 μg/ml, wherein the concentration refers to the weight of all active ingredients per ml of the composition. In embodiments, the composition may have a concentration of about 0.2 μg/ml to about 140 μg/ml, or about 0.2 μg/ml to about 130 μg/ml, or about 0.2 μg/ml to about 120 μg/ml, or about 0.2 μg/ml to about 110 μg/ml, or about 0.2 μg/ml to about 100 μg/ml, or about 0.2 μg/ml to about 90 μg/ml, or about 0.2 μg/ml to about 80 μg/ml, or about 0.2 μg/ml to about 70 μg/ml, or about 0.2 μg/ml to about 60 μg/ml, or about 0.2 μg/ml to about 50 μg/ml, or about 0.2 μg/ml to about 40 μg/ml, or about 0.2 μg/ml to about 30 μg/ml, or about 0.2 μg/ml to about 20 μg/ml, or about 0.2 μg/ml to about 10 μg/ml, or about 0.2 μg/ml to about 5 μg/ml, or about 0.2 μg/ml to about 4 μg/ml, or about 0.3 μg/ml to about 4 μg/ml, or about 0.4 μg/ml to about 4 μg/ml, or about 0.5 μg/ml to about 4 μg/ml, or about 0.6 μg/ml to about 4 μg/ml, or about 0.7 μg/ml to about 4 μg/ml, or about 0.8 μg/ml to about 4 μg/ml, or about 0.9 μg/ml to about 4 μg/ml, or preferably about 1.0 μg/ml to about 4 μg/ml. Advantageously, it was found that, due to the synergism between the oligomer of Formula (I) and the anti-fungal agent, at concentrations as low as 1.5 μg/ml, the composition may still effectively inhibit growth of microbes such as Candida albicans.

The composition as described herein may comprise at least one oligomer of Formula (I) and at least one anti-fungal agent. The anti-fungal agent and the oligomer of Formula (I) may be provided at weight ratio of about 2:1 to 1:1500, or about 1:1 to 1:1500, or about 1:2 to 1:1500, or about 1:4 to 1:1500, or about 1:6 to 1:1500, or about 1:8 to 1:1500, or about 1:10 to 1:1500, or about 1:15 to 1:1500, or about 1:20 to 1:1500, or about 1:30 to 1:1500, or about 1:40 to 1:1500, or about 1:50 to 1:1500, or about 1:60 to 1:1500, or about 1:70 to 1:1500, or about 1:80 to 1:1500, or about 1:90 to 1:1500, or about 1:100 to 1:1500, or about 1:150 to 1:1500, or about 1:200 to 1:1500, or about 1:250 to 1:1500, or about 1:300 to 1:1500. In one embodiment, the oligomer of Formula (I) and the anti-fungal agent may be provided at a weight ratio of about 1:333.

The weight ratio of the oligomer of Formula (I) and the anti-fungal agent may also be provided at about 1:350 to 1:1500, or about 1:400 to 1:1500, or about 1:450 to 1:1500, or about 1:500 to 1:1500, or about 1:550 to 1:1500, or about 1:600 to 1:1500, or about 1:650 to 1:1500, or about 1:700 to 1:1500, or about 1:750 to 1:1500, or about 1:800 to 1:1500, or about 1:850 to 1:1500, or about 1:900 to 1:1500, or about 1:950 to 1:1500, or about 1:1000 to 1:1500, or about 1:1000 to 1:1500, or about 1:1100 to 1:1500, or about 1:1200 to 1:1500, or preferably about 1:1200 to 1:1400. In another embodiment, the weight concentration ratio of the anti-fungal agent and the oligomer of Formula (I) may be 1:1333.

Advantageously, the composition as disclosed herein shows improved anti-microbial activity as compared to the oligomer of Formula (I) alone. In particular, the composition demonstrates improved anti-fungal effects as compared to the oligomer of Formula (I) alone or the triazole anti-fungal agent alone. Without being bound by theory, the synergistic effect of the described composition may arise from the combined fungicidal action of between the oligomer of Formula (I) and fungistatic action of the triazole anti-fungal agent.

Surprisingly, the synergistic effect between the oligomer of Formula (I) and the triazole anti-fungal agent may be observed for oligomers which have n values of less than 5. These oligomers may be oligomers where n=1-4. These oligomers may comprise less than 6 units of [DABCO]²⁺, imidazolium and [TMEDA]²⁺. The synergistic interaction between the oligomer and the triazole anti-fungal agent may be measured through the fractional inhibitory concentration (FIC) index.

The FIC values of the composition comprising an oligomer of formula (I), wherein n is less than 5, may be less than 0.5, or about 0.1 to about 0.5, or about 0.1 to about 0.4, or preferably about 0.1 to about 0.3.

The MIC or MIC₅₀ values of the oligomer of Formula (I) may be reduced when used in a composition comprising a triazole-based anti-fungal agent. In embodiments, the MIC₅₀ value of the oligomer when used in the composition may be reduced by at least 50%, as compared to the MIC₅₀ value of the oligomer when used alone. In some embodiments, the MIC₅₀ value of the oligomer may be reduced by about 50-90%, or about 55-90%, or about 60-90%, or about 65-90%, or about 70-90%, or about 75-90%, or preferably about 80-90%, as compared to the MIC₅₀ of the oligomer when used alone. In preferred embodiments, the MIC₅₀ value of the oligomer is reduced by about 87.5% compared to the MIC₅₀ value of the oligomer when used alone.

The MIC value of the anti-fungal agent may also be reduced when used in combination with the oligomer of Formula (I). In embodiments, the MIC₅₀ of the anti-fungal agent in the composition may be reduced by at least 50% compared to the MIC₅₀ of the anti-fungal agent when used alone. In some embodiments, the MIC₅₀ value of the oligomer may be reduced by about 50-99%, or about 55-99%, or about 60-99%, or about 65-99%, or about 70-99%, or about 75-99%, or about 80-99%, or about 85-99%, or preferably about 90-99%, compared to the MIC₅₀ of the anti-fungal agent when used alone. In preferred embodiments, the MIC₅₀ value of the anti-fungal agent may be reduced by about 94% as compared to the MIC₅₀ value of the anti-fungal agent when used alone.

In one embodiment, the FIC index of a composition comprising an oligomer, wherein n=3; and fluconazole is about 0.38. The oligomer which demonstrates a FIC index of about 0.38 with the anti-fungal agent may comprise n-octyl terminal E groups, three [R₁-L] units and an imidazolium R₂ group. The first [R₁-L] unit may comprise an imidazolium R₁ group and a para-xylylene linker; the second [R₁-L] unit may comprise a [DABCO]²⁺ R₁ group and a trans-butenyl linker; while the third [R₁-L] unit may comprise a [DABCO]²⁺ unit and a ₚₐᵣₐ-xylylene linker. The weight concentration ratio of the anti-fungal agent to the oligomer may be 1:2

In other embodiments, the composition comprising an oligomer wherein n=3 and fluconazole may have an FIC index of about 0.19 to 0.25. The oligomer of the composition may comprise n-octyl terminal E groups, three [R₁-L] units and an imidazolium R₂ group. The first [R₁-L] unit may comprise an imidazolium R₁ group and a para-xylylene linker]; while the second and third [R₁-L] units may each comprise a [DABCO]²⁺ R₁ group and a para-xylylene linker. The anti-fungal agent and oligomer may be provided at a weight concentration ratio of 1:8. The MIC₅₀ value of the oligomer when used in the composition may be 2 μg/ml; while the MIC₅₀ value of the anti-fungal agent in the composition may be 0.25 μg/ml. The composition may be provided at a combined concentration of 2.25 μg/ml.

Advantageously, oligomers with 3 [R₁-L] units, wherein the second [R₁-L] unit is the comprises a trans-butenyl L group may demonstrate better anti-microbial and more specifically, better anti-fungal activity as compared to oligomers of the same length, with a central o-xylylene or p-xylylene linker. Further advantageously, experimental results indicate that oligomers with 3 [R₁-L] units, wherein the L linker in the second [R₁-L] unit is trans-2-butenyl unit may be fungicidal.

Without being bound by theory, it is thought that the synergistic effect may be influenced by the interaction between the anti-fungal agent and the oligomer of Formula (I). It may be postulated that three types of mechanisms may contribute to the synergistic effect between two or more antifungal agents. The first mechanism may involve the sequential inhibition of a common biochemical pathway at different steps. The second proposed mechanism may be the simultaneous inhibition of cell wall and cell membrane targets in fungi. The third mechanism may be the simultaneous action of the first anti-fungal agent on the cell wall or cell membrane to enhance the penetration of a second antifungal agent.

Molecular dynamics simulations suggest that oligomers wherein n is less than 5 may have labile interactions with the triazole anti-fungal agents; while oligomers with n=5 may couple strongly to the fluconazole molecules. Accordingly, it may be postulated that the labile interaction between the oligomer and the anti-fungal agent may facilitate the synergistic effect of the composition. In particular, it may be proposed that such interactions may allow oligomers to attack the cell wall and/or cell membrane which may facilitate the passage of the antifungal agent into the cell matrix. This may improve the anti-fungal effect of the composition.

In another aspect of the disclosure, there is provided a method of preparing an oligomer of Formula (I). The method may comprise the steps of i) contacting a starting material of the formula [E-R₁-(L-R₁)ₐ] with [Br-L-Br] to obtain a first reaction product under a first set of reaction conditions, and ii) optionally, contacting the first reaction product with (R₂-L)_{b}-R₂ under a second set of reaction conditions. The R₁ group may be a positively charged diammonium group or a positively charged N-heterocycle. The positively charged N-heterocycle may be a 5- or 6-membered N-heterocycle. The R₁ group may in each instance be independently selected from the group consisting of [DABCO]²⁺, imidazolium or [TMEDA]²⁺. The structure of the R₁ groups is shown below:

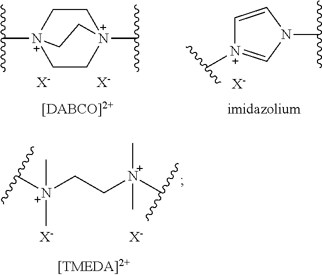

R₁ may also comprise an anion X, which in each instance, may be the same or different. Anion X may be a monoatomic or polyatomic anion. Anion X may preferably be a halide such as fluoride, chloride, bromide or iodide, preferably chloride or bromide. In preferred embodiments, X may be a bromide anion.

L may, in each instance, be independently selected from the group consisting of optionally substituted alkyl, optionally substituted alkenyl, optionally substituted alkynyl, optionally substituted aryl, optionally substituted arylalkyl, optionally substituted arylalkenyl, optionally substituted arylalkynyl, optionally substituted alkylaryl, optionally substituted alkenylaryl, and optionally substituted alkynylaryl.

In embodiments, L may be an optionally substituted alkenyl, optionally substituted aryl, optionally substituted arylalkenyl or optionally substituted alkylaryl group, preferably an optionally substituted alkylaryl group. In some embodiments, L may be an aryl group comprising two alkyl substituents, or an alkenyl group comprising 2 alkyl substituents. In some embodiments, L may be para-xylylene or ortho-xylylene. In other embodiments, L may be trans-2-butenyl.

E may be independently selected from the group consisting of: optionally substituted alkyl, optionally substituted aryl, optionally substituted arylalkyl and optionally substituted alkylaryl; and is between 2 to 20 carbon atoms in length. In embodiments, E may be an optionally substituted alkyl or optionally substituted aryl group, preferably an optionally substituted alkyl group. In some embodiments, the E terminal group may be an optionally substituted alkyl group comprising 8 carbon atoms. In preferred embodiments, E may be a n-octyl group.

The value of a and b may independently be either 0 or 1.

The starting materials [E-R₁-(L-R₁)ₐ] and [Br-L-Br] may be provided at a molar ratio of about 3:1 to about 1:7, or about 3:1 to about 1:6, or about 3:1 to about 1:5. The molar ratio of the starting materials may depend on the desired first reaction product. In embodiments, the first reaction product may have the formula [E-R₁-(L-R₁)ₐ-L-(R₁-L)ₐ-R₁-E] or [E-R₁(L-R₁)ₐ-L-Br].

In embodiments, a first reaction product obtained from step (i) may have the formula [E-R₁-(L-R₁)ₐ-L-(R₁-L)ₐ-R₁-E]. The molar ratio of the [E-R₁-(L-R₁)ₐ] and [Br-L-Br] starting materials may be from 3:1 to 2:1, preferably about 2.5:1. Oligomers of n=1 may be obtained directly by the method of step (i).

In other embodiments, a first reaction product obtained from step (i) may be of the formula [E-R₁(L-R₁)ₐ-L-Br]. The molar ratio of the [E-R₁-(L-R₁)ₐ] and [Br-L-Br] starting materials may be from about 1:1 to 1:7, or about 1:1 to 1:6, or preferably about 1:1 to about 1:5. This method may be used to obtain oligomers where n is more than 1.

Step (i) of method of preparing the oligomer of Formula (I) as described herein may be carried out in a polar, aprotic organic solvent. Non-limiting examples of such solvents may include dimethylformamide, tetrahydrofuran, dimethylsulfoxide, acetonitrile, ethyl acetate, dichloromethane and acetone. In embodiments, step (i) may be carried out using dimethylformamide or acetonitrile as a solvent.

Step (i) of the method of preparing the oligomer of Formula (I) as described herein may be carried out at a temperature of about 30 to 150° C., or about 30 to 140° C., or about 30 to 130° C., or about 30 to 120° C., or about 30 to 110° C., or about 30 to 100° C., or about 40 to 100° C., or about 50 to 100° C. or about 60 to 100° C., or about 70 to 100° C., or preferably about 80 to 100° C. In embodiments, step (i) may be carried out at 90° C.

Step (i) of the method of preparing the oligomer of Formula (I) as described herein may be carried out for a duration of at least 6 hours, or about 6 to 48 hours, or about 6 to 46 hours, or about 6 to 44 hours, or about 6 to 42 hours, or about 6 to 40 hours, or about 6 to 38 hours, or about 6 to 36 hours, or about 8 to 36 hours, or about 10 to 36 hours, or about 12 to 36 hours, or about 14 to 36 hours, or about 16 to 36 hours, or about 18 to 36 hours, or about 20 to 36 hours, or about 22 to 48 hours, or preferably about 24 to 48 hours.

The method of preparing oligomers where n is more than 1 may further comprise a second step (step ii). Step (ii) may comprise contacting the first reaction product with a compound of the formula [(R₂-L)_{b}-R₂] under a second set of reaction conditions.

R₂, in each instance, may be a positively charged alkyl diamine or a positively charged N-heterocycle. The N-heterocycle may be a 5- or 6-membered N-heterocycle. The N-heterocycle may be an aromatic N-heterocycle. R₂, in each instance, may be independently selected from the group consisting of [DABCO]²⁺, imidazolium and [TMEDA]²⁺. In embodiments, R₂ may be an imidazolium group.

R₂ may also comprise an anion X, which in each instance, may be the same or different. Anion X may be a monoatomic or polyatomic anion. Anion X may preferably be a halide such as fluoride, chloride, bromide or iodide, preferably chloride or bromide. In preferred embodiments, X may be a bromide anion.

The molar ratio of [(R₂-L)_{b}-R₂] to the first reaction product may be about 1:2 to about 1:10, or about 1:2 to about 1:9, or about 1:2 to about 1:8, or about 1:2 to about 1:7, or about 1:2 to about 1:6, or about 1:2 to about 1:5, or about 1:2 to about 1:4, or preferably about 1:3 to about 1:4.

The solvent in step (ii) may be a polar, protic solvent. Non-limiting examples of polar protic or solvents may include methanol, ethanol, isopropanol, water and formic acid. In embodiments, step ii) may carried out using methanol.

Step (ii) may be carried out at a temperature of 30 to 150° C., or about 30 to 140° C., or about 30 to 130° C., or about 30 to 120° C., or about 30 to 110° C., or about 30 to 100° C., or about 30 to 90° C., or about 30 to 80° C., or about 30 to 70° C., or about 30 to 60° C., or preferably about 30 to 50° C. In embodiments, step (ii) may be carried out at 40° C.

Step (ii) of the method of preparing the oligomer of Formula (I) as described herein may be carried out for a duration of at least 6 hours, or about 6 to 60 hours, or about 8 to 60 hours, or about 10 to 60 hours, or about 12 to 60 hours, or about 14 to 60 hours, or about 16 to 60 hours, or about 18 to 60 hours, or about 20 to 60 hours, or about 22 to 60 hours, or about 24 to 60 hours, or about 24 to 58 hours, or about 24 to 56 hours, or about 24 to 54 hours, or about 24 to 52 hours, or about 24 to 50 hours, or about 26 to 50 hours, or about 28 to 50 hours, or about 30 to 50 hours, or about 32 to 50 hours, or about 34 to 50 hours, or preferably about 36 to 80 hours.

### EXAMPLES

### Example 1—Synthesis of the Oligomers

### General Information

All solvents were purchased from Sigma-Aldrich and used without further purification. All other reagents were used as received, except where otherwise noted in the experimental text.

### List of Abbreviations

THF—tetrahydrofuran

MeCN—Acetonitrile

MeOH—Methanol

DMF—Dimethylformamide

DMSO—Dimethylsulfoxide

DABCO—1, 4-diazabicyclo[2.2.2]octane

TMEDA—tetramethylethylenediamine

eq. —molar equivalents

MHB—Mueller-Hinton Broth

YMB—Yeast Mold Broth

CFU—Colony Forming Units

ATCC—American Type Cell Culture

OD—Optical Density

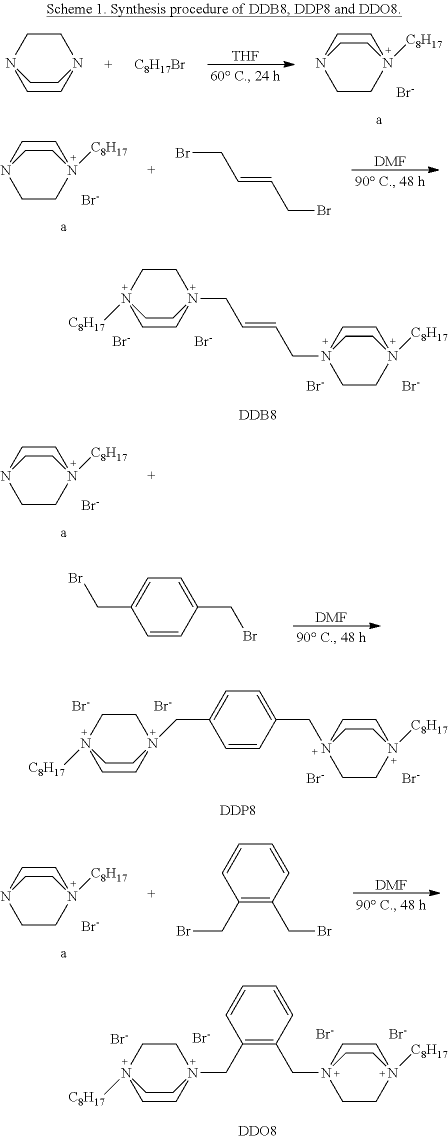

### Example 1.1—Synthesis of a

A solution of bromooctane (1.0 eq, in THF) was added dropwise to a solution of 1,4-diazabicyclo[2.2.2]octane (DABCO, 5.0 eq, in THF) at 60° C. After 24 hours, THF was removed under vacuum and the resulting white solids were washed with diethyl ether. a was obtained quantitatively as colorless liquid (>95% yield).

### Example 1.2—Synthesis of DDB8, DDP8 and DDO8

A solution of a (2.5 eq, in DMF) was mixed with a solution of trans-1,4,-dibromobut-2-ene (1.0 eq, in DMF). After stirring at 60° C. for 48 hours, DMF was removed under vacuum and the resulting white solids were washed with acetone and then diethyl ether. DDB8 was obtained quantitatively as white solid. The synthesis of DDP8 and 0008 was similar to DDB8.

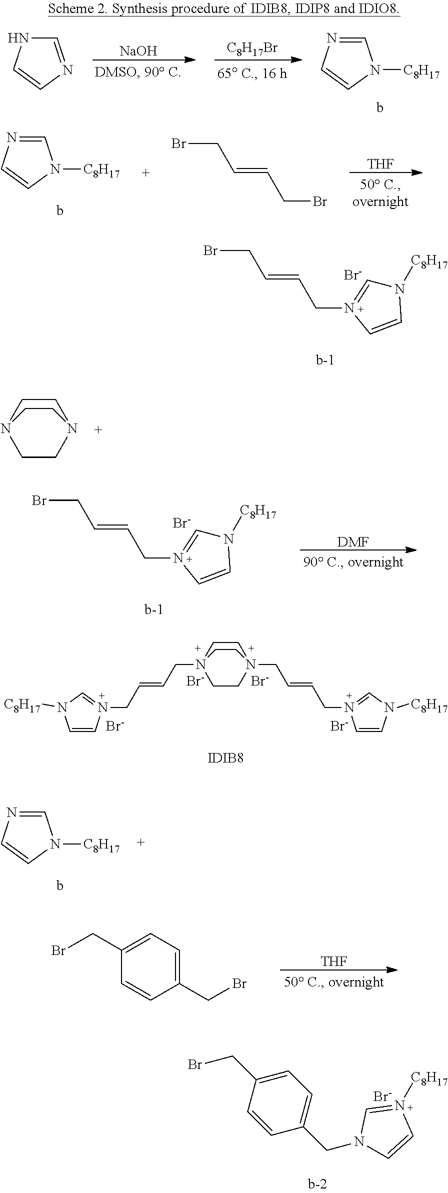

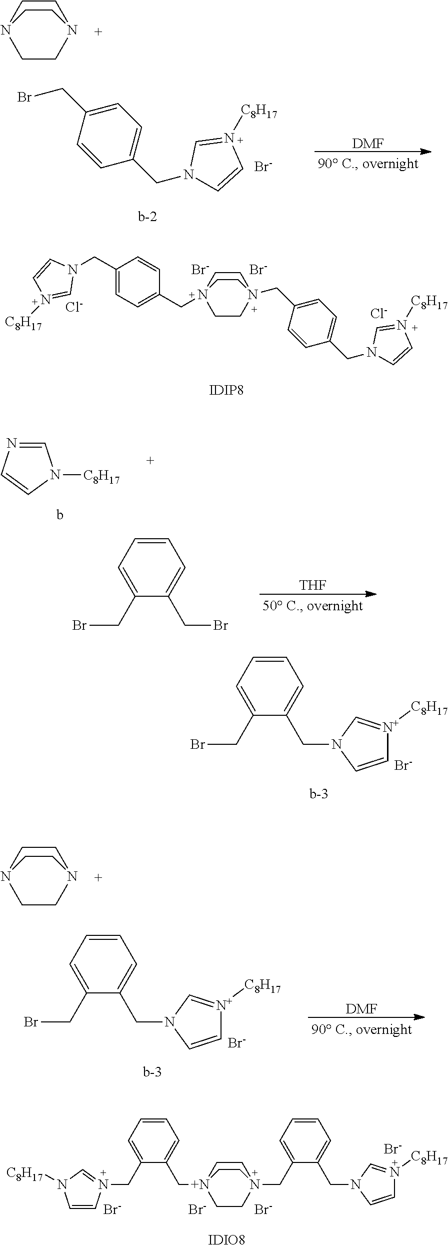

### Example 1.3—Synthesis of b

A mixture of imidazole (1.0 eq) and sodium hydroxide (1.0 eq) in DMSO was heated to 90° C. for 2 hours, and then cooled to room temperature. A solution of 1-bromooctane (1.0 eq) in DMSO was added dropwise to the mixture. After stirring at room temperature for 3 hours, the mixture was heated up to 65° C. for 16 hours with constant stirring. The solution obtained was mixed with water and then extracted with diethyl ether several times. Diethyl ether was removed under vacuum and b was obtained as a yellow liquid.

### Example 1.4—Synthesis of b-1

A solution of b (1.0 eq, 1.80 g, 10.0 mmol in THF) was added dropwise to a solution of trans-1,4,-dibromobut-2-ene (2.0 eq, 0.12 g in THF) at 70° C. After overnight stirring, THF was removed from the solution under vacuum. The obtained liquid was washed with diethyl ether 3 times and then dried under vacuum (1.15 g, 29%). The synthesis of b-2 and b-3 was similar to b-1.

### Example 1.5—Synthesis of IDIB8

A mixture of b-1 (3.0 eq) and DABCO (1.0 eq) was stirred in DMF at 80° C. After overnight stirring, DMF was removed from the solution under vacuum. The obtained liquid was washed with acetone, and then dried under vacuum. The synthesis of IDIP8 and 10108 was similar to IDIB8.

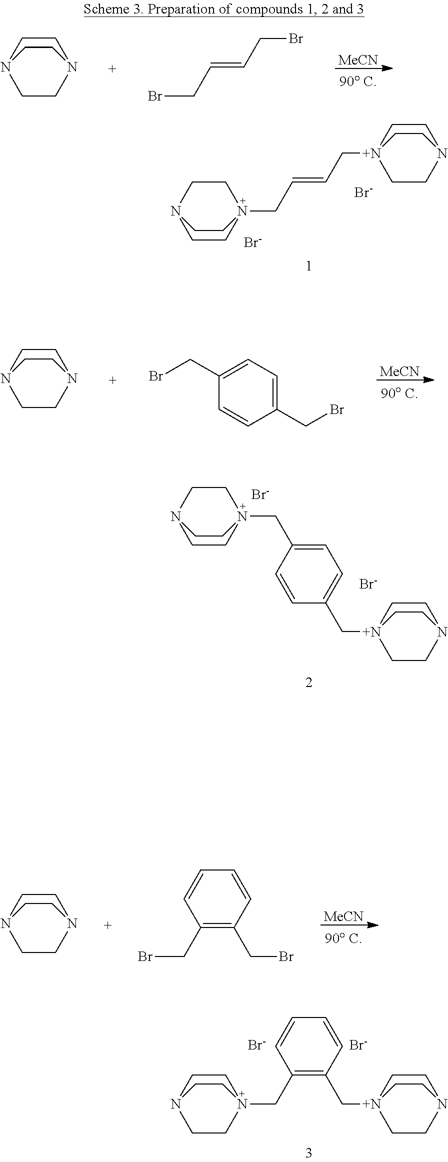

### Example 1.6—Synthesis of 1, 2 and 3

A solution of DABCO (8.0 eq) was dissolved in MeCN and heated to 80° C. and a solution of α,α′-Dibromo-p-xylene (1.0 eq) or trans-1,4,-dibromobut-2-ene (1.0 eq) was added dropwise to it. The resulting solution was stirred at 90° C. for 24 hours. The solids were collected and washed with MeCN, followed by ethyl acetate, and then diethyl ether to obtain 1 as a white powder. The synthesis of 2 and 3 was similar to 1.

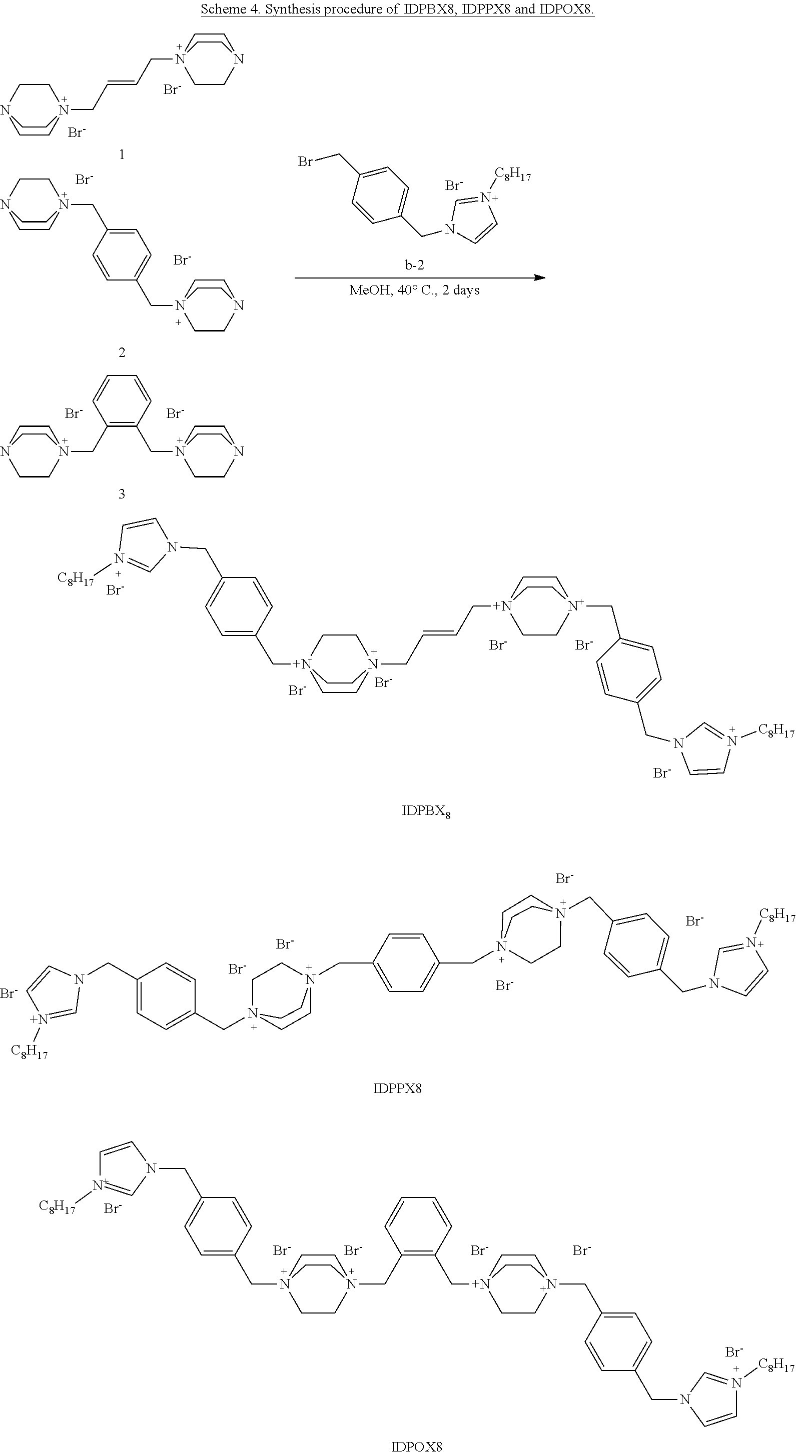

### Example 1.7—Synthesis of IDPBX8

1 (1.0 eq) was dissolved in MeOH and heated to 60° C. b-2 (4.0 eq) dissolved in MeOH was added to the solution of 1. The mixture was stirred constantly at 40° C. for 2 days. MeOH was then removed under vacuum. The resulting white solids were washed with acetone and then dried to obtain IDPBX8.

### Example 1.8—Synthesis of IDPPX8 and IDPDX8

The synthesis of IDPPX8 and IDPDX8 was similar to IDPBX8.

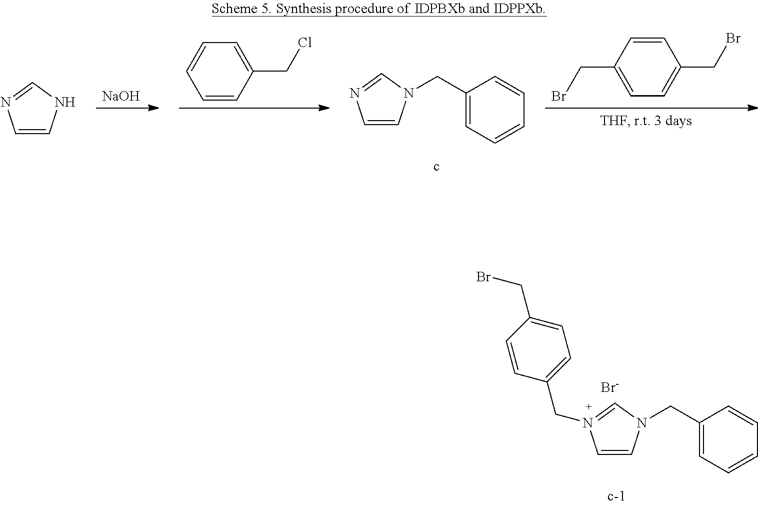

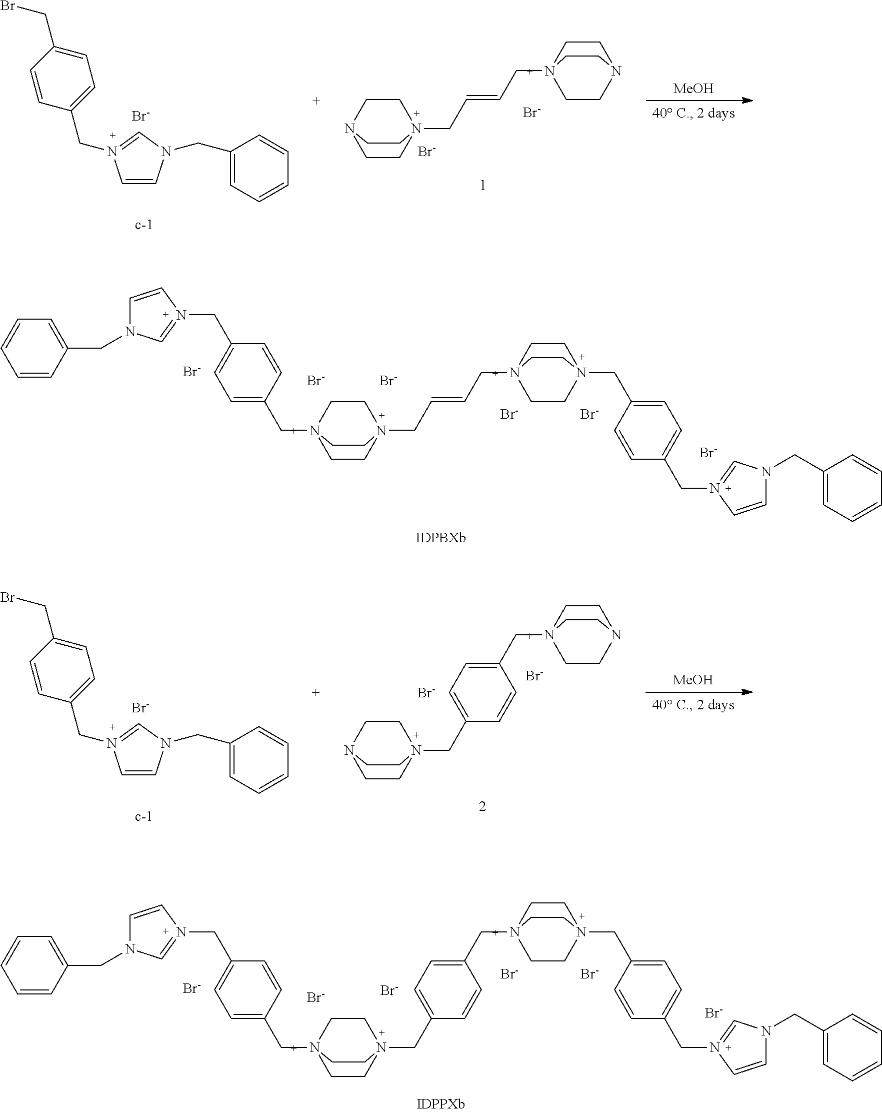

### Example 1.9—Synthesis of c

The synthesis of c was similar to the synthesis of b except that c was obtained as colorless crystals.

### Example 1.10—Synthesis of c-1

The synthesis of c-1 was similar to the synthesis of b-2. A mixture of c (1.0 eq) and α,α′-Dibromo-p-xylene (5.0 eq) was stirred in THF at room temperature for 3 days. The mixture was concentrated by removing THF under vacuum. The obtained solid/liquid was washed with diethyl ether and then extracted with acetone. Acetone was removed under vacuum. c-1 was obtained as light yellow liquid.

### Example 1.11—Synthesis of IDPBXb and IDPPXb

The same procedure as the synthesis of IDPBX8 was used to synthesize IDPBXb and IDPPXb.

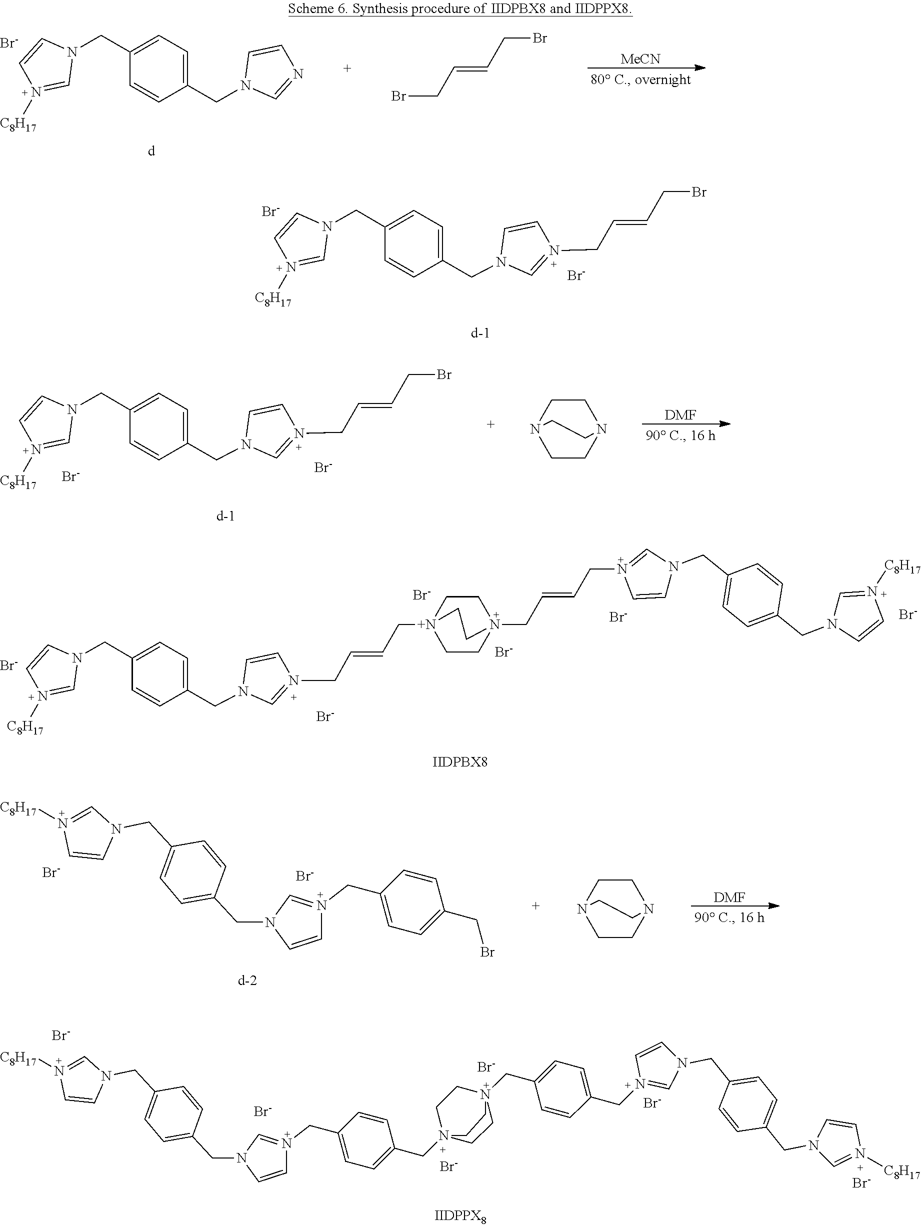

### Example 1.12—Synthesis of d-1

The synthesis of d was reported previously (Riduan et al, Small, 2016, 12, pages 1928-1934). A solution of d (1.0 eq) in acetonitrile was added dropwise to a solution of trans-1,4-dibromo-2-butene (7.0 eq). The resulting mixture was stirred at 80° C. overnight. Solvent was removed under vacuum and the resulting solids were washed with ethyl acetate. d-1 was obtained as light yellow liquid.

### Example 1.13—Synthesis of IIDPBX8 and IIDPPX8

A solution of d-1 (3.0 eq) in DMF was mixed with a solution of DABCO (1.0 eq). The resulting mixture was stirred at 90° C. for 16 h. After reaction, the solvent was removed under vacuum. After washing with acetone, the product was dried under vacuum. IIDPBX8 was obtained as white powder.

The synthesis of d-2 was reported previously (Yuan et al, ChemMedChem, 2017, 12, pages 835-840). IIDPPX8 was synthesized under similar condition as IIDPBX8.

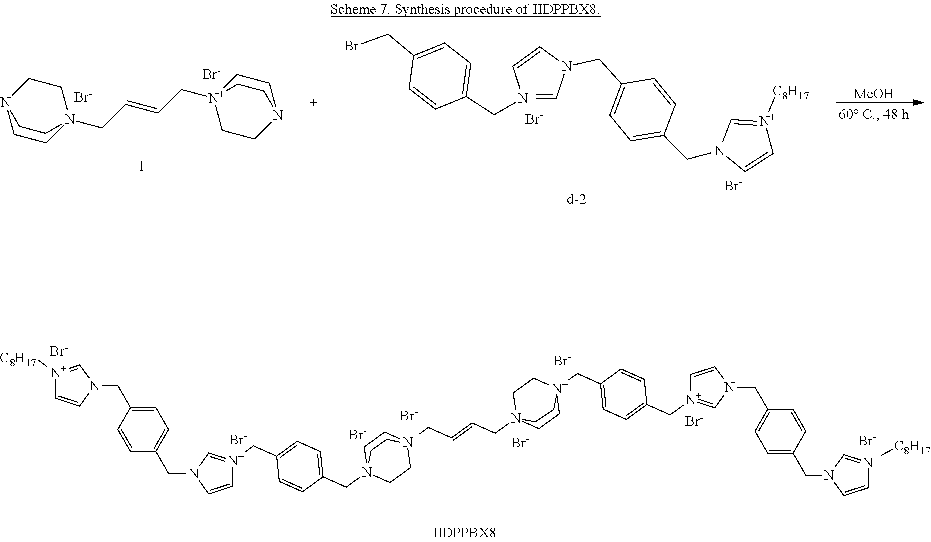

### Example 1.14—Synthesis of IIDPPBX8

A solution of 1 (1.0 eq) in MeOH was mixed with a solution of d-2 (5.0 eq). The mixture was stirred constantly at 60° C. for 48 hours. MeOH was then removed under vacuum and the resulting solids were washed with acetone and then DMF. The resulting solids were dried under vacuum. IIDPPBX8 was obtained as white solids.

### Example 2—Minimum Inhibitory Concentration

Staphylococcus aureus (ATCC 6538, Gram-positive), Escherichia coli (ATCC 8739, Gram-negative), Pseudomonas aeruginosa (ATCC 9027, Gram-negative), and Candida albicans (ATCC 10231, fungus) were used as representative microorganisms to challenge the antimicrobial functions of the oligomers. All bacteria and fungus were frozen at −80° C., and were grown overnight at 37° C. in Mueller Hinton Broth (MHB, BD Singapore) prior to experiments. Fungus was grown overnight at 22° C. in Yeast Mold Broth (YMB, BD Singapore). Subsamples of these cultures were grown for a further 3 h and diluted to give an optical density (O.D.) value of 0.07 at 600 nm (OD600=0.07), corresponding to 3×10⁸ CFU mL⁻¹ for bacteria and 10⁶ CFU mL⁻¹ for fungus (McFarland's Standard 1; confirmed by plate counts).

The oligomers were dissolved in MHB at a concentration of 4 mg mL⁻¹ and the minimal inhibitory concentrations (MICs) were determined by microdilution assay (Niimi et al, Odontology, 2010, 98, pages 15-25). Bacterial solutions (100 μL, 3×10⁸ CFU mL⁻¹) were mixed with 100 μL of oligomer solutions (normally ranging from 4 mg mL⁻¹ to 2 μg mL⁻¹ in serial two-fold dilutions) in each well of the 96-well plate. The plates were incubated at 37° C. for 24 h with constant shaking speed at 300 rpm. The MIC measurement against Candida albicans was similar to bacteria except that the fungus solution was plated at ˜10⁶ CFU mL⁻¹ in YMB and the plates were incubated at room temperature.

The MICs were taken as the concentration of the oligomer at which less than 50% microbial growth was observed with the microplate reader (TECAN). Medium solution containing microbial cells alone were used as control (100% microbial growth). The assay was performed in four replicates and the experiments were repeated at least two times.

### Example 3—Checkerboard Assay

In order to evaluate whether individual oligomer compounds exhibit synergy or indifference in combination with fluconazole against C. albicans, checkerboard assays were performed as described previously with slight modification (Singh et al, Am. J. Physiol. Lung Cell Mol. Physiol., 2000, 279, L799-L805). Two-fold serial dilution of oligomers and fluconazole were prepared in YMB at 4 times the strength of the final concentration ranging from 1/16 to two times of the MIC. Aliquots of 50 μL of each component at a concentration of 4 times the targeted final concentration were mixed in a 96-well plate. A row and a column in which a serial dilution of each agent was present alone were also prepared for MIC test. Then each solution in the well plate was inoculated with 100 μL of logarithmically grown 106 cells/ml C. albicans cells in the 96-well plate. The plate also contained a column with C. albicans only as control (100% cell growth). The cells were incubated at room temperature for 24 h with constant shaking, after which cell growth (i.e. the O.D. at 600 nm) was monitored with a microplate reader (TECAN)

Table 1 shows the MIC values of the oligomers when tested against Staphylococcus aureus (S.A.), Pseudomonas aeruginosa (P.A.), Escherichia coli (E.C.) and Candida albicans (C.A). The MIC was tested against ˜10⁶ CFU/ml of microbes, and the MIC was taken to be the lowest concentration of the antimicrobial oligomer at which no visible growth was observed by unaided eyes. In Table 1, the MIC₅₀ value, i.e. the concentration of the oligomer which may inhibit 50% fungal growth is indicated in brackets. The MIC values of the oligomers were compared against other imidazolium polymers, IBN-C8 and IBN132b, the structures of which are depicted below.

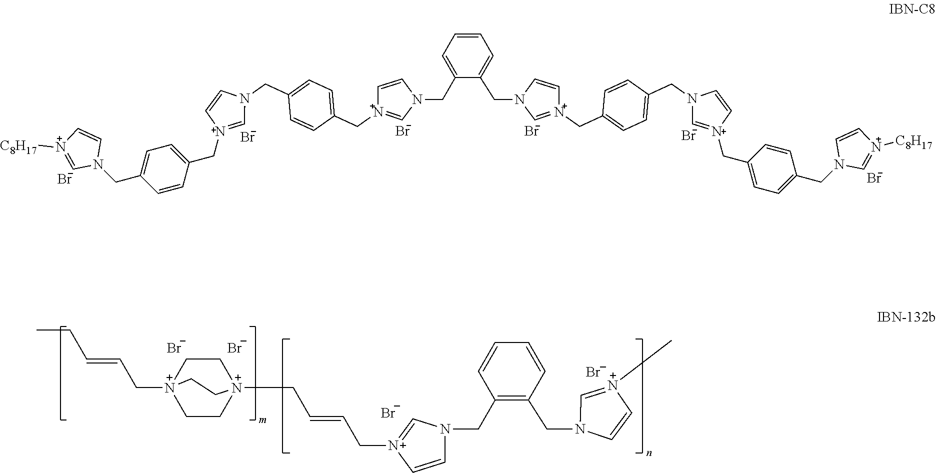

TABLE 1
Antimicrobial activity (MIC, μg/ml), the fractional inhibitory concentration
index (FIC) with Fluconazole, hemolytic property (HC10, μg/ml) and critical
micelle concentration (CMC, μg/ml) of the ammonium imidazolium oligomers.

| | Sample | MIC (μg/ml)^{a} | | | | | HC₁₀ | | CMC |
|---|---|---|---|---|---|---|---|---|---|
| n | name | S.A. | E.C. | P.A. | C.A. | | (μg/ml) | FIC | (μg/ml) |
| 1 | DDB8 | 4 | 8 | 125 | 125 | (16) | >2000 | 0.25 | 1552 |
| | DDP8 | 16 | 16 | 2000 | 500 | (31) | >2000 | 0.19 | 1896 |
| | DDO8 | 4 | 8 | 62 | 125 | (16) | >2000 | 0.38 | 3038 |
| 2 | IDIP8 | 2 | 16 | 1000 | 125 | (8) | >2000 | 0.25 | 985 |
| | IDIB8 | 8 | 16 | 500 | >500 | (32) | >2000 | 0.25 | 1829 |
| | IDIO8 | 8 | 16 | 250 | 62 | (32) | >2000 | 0.25 | 1667 |
| 3 | IDPBX8 | 16 | 31 | 62 | 8-31 | (2-8) | >2000 | 0.38 | 1456 |
| | IDPPX8 | 8 | 62 | 500 | 62 | (8-31) | >2000 | 0.25 | 1552 |
| | IDPOX8 | 4 | 8 | 62 | 125 | (16) | >2000 | 0.25 | 1367 |
| | IDPBXb | 125 | 62 | >1000 | >1000 | (1000) | >2000 | na | 1732 |
| | IDPPXb | 125 | 62 | >1000 | 2000 | (500) | na | 0.5 | 1860 |
| | ITPPX8 | 4 | 16 | 500 | 125 | (16) | >2000 | 0.25 | 1342 |
| 4 | IIDPBX8 | 4 | 62 | 250 | 62 | (8) | >2000 | 0.31 | 291 |
| | IIDPPX8 | 4 | 16 | 125 | 125 | (16) | >2000 | 0.38 | 451 |
| 5 | IIDPPBX8 | 8 | 31 | 125 | 31 | (2-8) | >2000 | 1.00 | 727 |

| | IBN-C8 | 4 | 8 | 16 | 16 | >2000 | 2.00 | 226 |
|---|---|---|---|---|---|---|---|---|
| copolymer | IBN-132-3^{b} | 16 | 16 | 62 | 2 | >2000 | 2.00 | 2076 |
| Azoles | Fluconazole | — | — | — | 2-4 | — | — | — |
| | Itraconazole | — | — | — | 0.016-0.03 | — | — | — |
| | Voriconazole | — | — | — | 0.025-0.1 | — | — | — |

Synergy between fluconazole and oligomers was determined by calculating the fractional inhibitory concentration index (FIC). FIC was calculated as follows: FIC=(MIC_{oligomer A in combination}/MIC_{oligomer A alone})+(MIC_{azole B in combination}/MIC_{azole B alone}). FIC values of 0.5, >0.5 to 4.0 and >4.0 indicated synergy, indifference, or antagonistic interactions for different combinations. The FIC values of the various oligomers with fluconazole are also shown in Table 1. The FIC of the oligomers with fluconazole was calculated using the lowest concentration of the oligomer that inhibited at least 50% grown of Candida albicans.

### Example 4—Synergistic Effect with Azoles Against Candida albicans

The interaction of selected oligomers with other triazoles such as itraconazole and voriconazole was also investigated using the checkerboard dilution method. For comparison, the interaction of the selected oligomer with a non-triazole antifungal agent norfloxacin was also investigated. The structure of norfloxacin is show below:

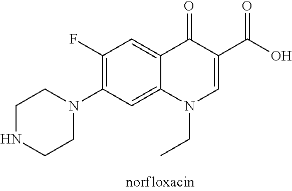 [SMILES: CCn1cc(C(=O)O)c(=O)c2cc(F)c(N3CCNCC3)cc21]

The MIC and FIC of different combinations of IDPBX8, IDPPX8 and IIDPPBX8 with the anti-fungal agents against Candida albicans are shown in Table 2.

TABLE 2
Effect of treatments with combinations of oligomers and triazoles
on the growth of Candida albicans according to the FIC.

| | | Alone MIC₅₀ | | Combined MIC₅₀ | | |
|---|---|---|---|---|---|---|
| Components | | (μg/ml) | | (μg/ml) | | |
| A | B | A | B | A | B | FIC |
| IDPBX8 | Fluconazole | 8 | 2 | 1 | 0.5 | 0.38 |
| IDPBX8 | Itraconazole | 8 | 0.03 | 1 | 0.015 | 0.62 |
| IDPBX8 | Voriconazole | 8 | 0.1 | 2 | 0.006 | 0.31 |
| IDPPX8 | Fluconazole | 16 | 4 | 2 | 0.25 | 0.19 |
| IDPPX8 | Itraconazole | 16 | 0.016 | 4 | 0.004 | 0.50 |
| IDPPX8 | Voriconazole | 16 | 0.025 | 4 | 0.003 | 0.30 |
| IIDPPBX8 | Fluconazole | 2 | 4 | 1 | 2 | 1.0 |

Synergy was also observed when IDPBX8 or IDPPX8 was applied together with voriconazole.

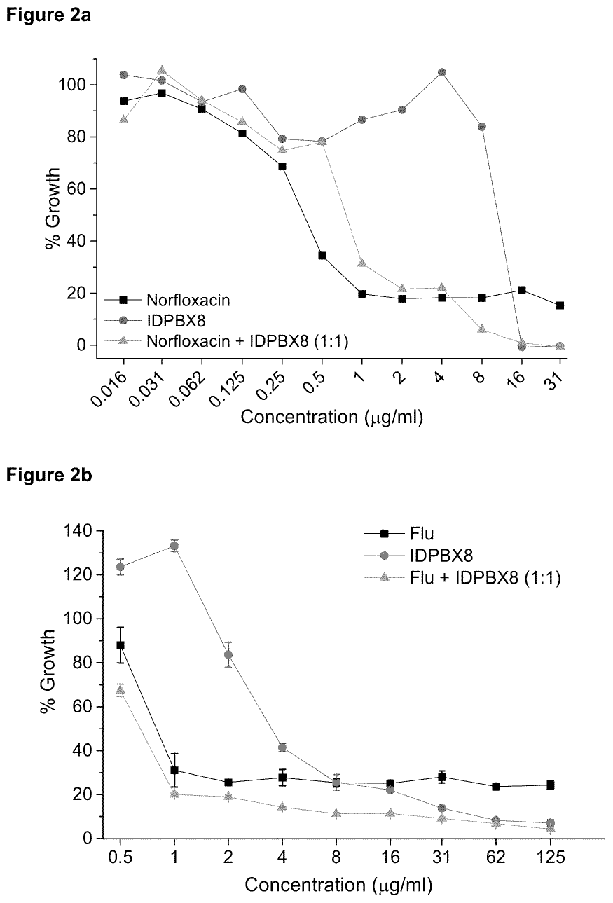

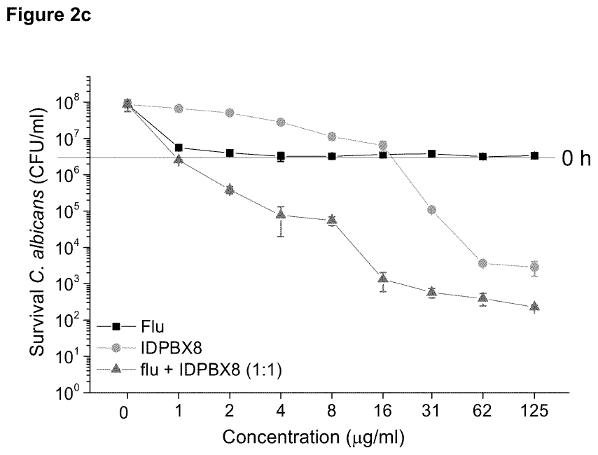

The growth of C. albicans in the presence of both IDPBX8 and fluconazole at different concentrations was also studied by recording the absorbance of the cell culture medium at 600 nm using a microplate reader. For comparison, the effect of IDPBX8 in combination with norfloxacin was also studied. The results are shown in FIGS. 2a and b.

Interestingly, the combination of IDPBX8 and norfloxacin (1:1 weight ratio) did not show improved efficacy while IDPBX8 combined with fluconazole showed higher activity than each compound/medicine used alone. The results of colony counting using nutrient agar plates reflected the concentration of survival C. albicans (FIG. 2c). Killing effect was observed even when the total concentration of the combination was 2 μg/ml.

### Example 5—Monitoring the Growth of C. albicans

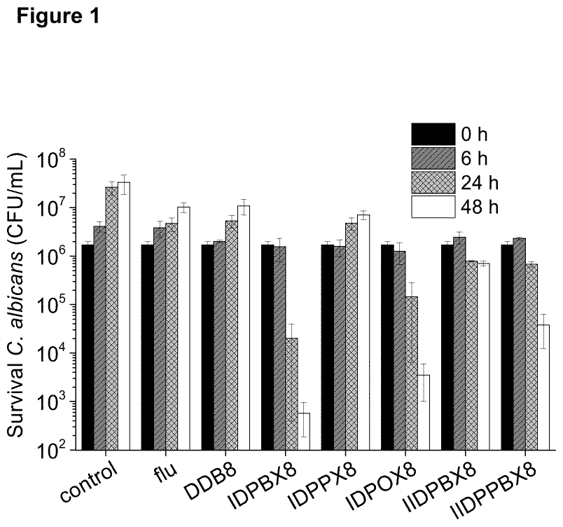

The killing efficacy of selected oligomers was evaluated against Candida albicans at a concentration of 62 μg/ml (FIG. 1). The antifungal activity of fluconazole was also measured for comparison.

The growth of C. albicans in the presence of IDPBX8 and fluconazole alone or their combination was monitored by measuring the absorbance at 600 nm with a microplate reader and quantified using colony counting methods. Briefly, material was dissolved in YMB (2 μg/mL to 62 μg/mL in serial two-fold dilution). A hundred microliters of each solution were placed into a 96-well microplate. Then 100 μg/mL of C. albicans suspension (7.6×10⁶ CFU/ml) was added into each well. So the final concentration of C. albicans was 3.8×10⁶ CFU/ml. Fungus growing in pure YMB was used as control. The 96-well plate was kept on a shaker at room temperature under constant shaking. After incubation for 24 h, the absorbance of the solutions (OD600) was measured with a plate reader (TECAN) and used to calculate the % growth using the absorbance of control solution as 100% growth. To quantify the number of viable fungi, after 24 h incubation, aliquots (100 μg/mL) were withdrawn and serially diluted with DPBS buffer (1:10). 100 μL of each dilution were spread onto nutrient agar plate (Luria-Bertani broth with 1.5% agar) and the colony forming units (CFU) were counted after incubation at room temperature for 2 days.

After 24 h treatment with fluconazole, the number of survival C. albicans was significantly less than the untreated control, indicating that this strain is susceptible to fluconazole. However, the quantity of survival C. albicans at 24 h was larger than the initially added C. albicans at 0 h, which means fluconazole is fungistatic rather than fungicidal. The viable C. albicans after 24 h treatment with oligomers were significantly less than the controls, implying that all of the synthetic oligomers are antifungal. Specifically, IDPPX8 and DDB8 are fungistatic while other oligomers are fungicidal. Although both IDPBX8 and IIDPPBX8 have the lowest MIC (2-8 μg/ml) against C. albicans, they showed different killing kinetics. IDPBX8 kills faster. 99% killing was observed after 24 h treatment and increased to 99.99% after 48 h.

### Example 6—Time-Kill Method

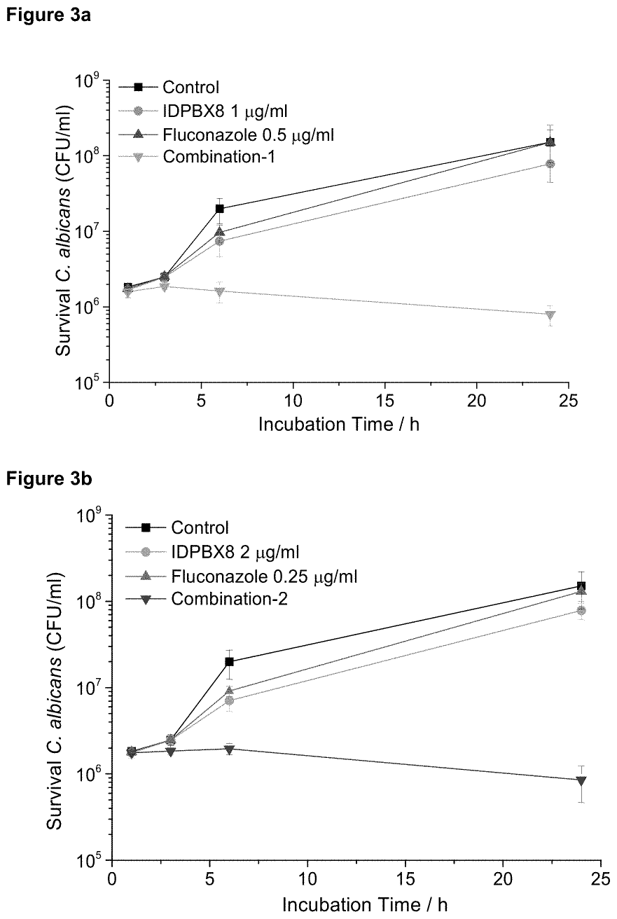

Time-kill experiments were performed with selected antifungal combinations according to the results of the checkerboard assay. IDPBX8 and fluconazole were tested alone and in combination at sub-MIC level (bellow original MIC values). The mixtures were inoculated with C. albicans adjusted to give a final concentration of about 10⁶ CFU/mL. After 1, 3, 6 and 24 h incubation at room temperature, the respective cell suspensions were collected (100 μL), serially diluted 1:10, and 100 μL of each dilution was spread on LB agar. Colonies were counted after 48 h incubation at room temperature and the CFU/mL was calculated accordingly. FIGS. 3a and 3b illustrate the synergistic kinetics of combinations of IDPBX8 and fluconazole in the time kill assay.

The combination of 1 μg/ml IDPBX8 and 0.5 μg/ml fluconazole, and of 2 μg/ml IDPBX8 and 0.25 μg/ml fluconazole revealed a stable and continuous inhibition of colony counts after 24 h compared to the single substance of IDPBX8 or fluconazole.

### Example 7—Resistance Studies

The method was adapted from that of Yuan and co-authors with modification (Yuan et al, Biomateri. Sci., 2019, 7, page 2317-2325). Drug resistance was induced by treating C. albicans repeatedly with IDPBX8, IDPBX8-fluconazole combination or fluconazole. First MICs of the tested compounds were determined against C. albicans using the broth microdilution method. Then serial passaging was initiated by transferring microbial suspension grown at the sub-MIC of the copolymers (¼ of MIC at that passage for C. albicans) for another MIC assay. After 24 h incubation, cells grown at the ¼-MIC of the test compounds were once again transferred and assayed for MIC. The MIC against C. albicans was tested for 35 passages.

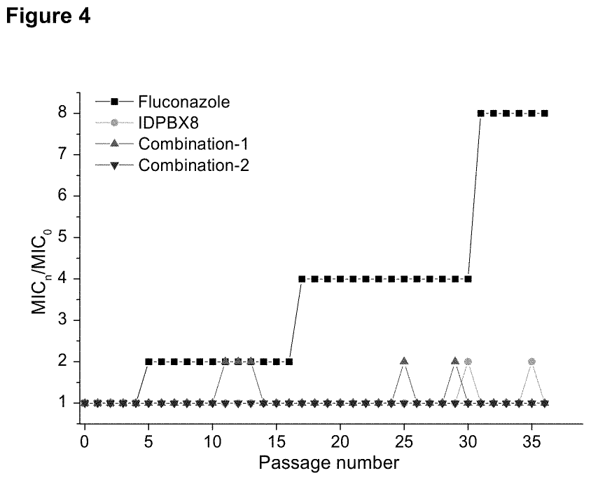

Two IDPBX8-fluconazole combinations were tested: IDPBX8 (2 μg/ml)+fluconazole (0.25 μg/ml); IDPBX8 (1 μg/ml)+fluconazole (0.5 μg/ml). Drug-resistant behavior was evaluated by recording the changes in the MIC normalized to that of the first passage (FIG. 4).

As shown in FIG. 4, the MIC of fluconazole against C. albicans increases at the 5th passage. By the 31th passage the MIC of fluconazole increases to 8 times of the original MIC. In contrast, the MICs of IDPBX8 are relatively stable over the entire 36 passages, indicating that no significant resistance was developed by C. albicans during the 36 consecutive days of treatment with IDPBX8.

### Example 8—Hemolysis Study

Fresh mouse red blood cells (RBCs) were diluted with PBS buffer to give an RBC stock suspension (4 vol % blood cells). 100 μL aliquots of RBC suspension were mixed with 100 μL oligomer solutions of various concentrations (ranging from 4 mg mL⁻¹ to 2 μg mL⁻¹ in serial two-fold dilutions in PBS). After 1 h incubation at 37° C., the mixture was centrifuged at 2000 rpm for 5 min. Aliquots (100 μl) of the supernatant were transferred to a 96-well plate. Hemolytic activity was determined as a function of hemoglobin release by measuring absorbance of the supernatant at 576 nm using a microplate reader. A control solution that contained only PBS was used as a reference for 0% hemolysis. Absorbance of red blood cells lysed with 0.5% Triton-X was taken as 100% hemolysis. The oligomer concentration which results in 10% hemolysis is indicated as HC₁₀ in Table 1. The data were expressed as mean and S.D. of four replicates.

% Hemolysis=[OD_{576 nm (polymer)}−OD_{576 nm (PBS)}]/[OD_{576 nm (Triton-X)}−OD_{576 nm (PBS)}]×100%

All the oligomers did not induce noticeable hemolysis. No hemolysis was observed even at 2000 μg/mL, the highest concentration we tested. Considered alongside their high antimicrobial activity, these oligomers are primarily qualified as active and non-toxic compounds which display high selectivity for a wide range of pathogenic microbes over mammalian cells.

### Statistical Analysis:

Data were expressed as means±standard deviation of the mean (S.D. is indicated by error bars). Student's t-test was used to determine significance among groups. A difference with p<0.05 was considered statistically significant.

### INDUSTRIAL APPLICABILITY

The present composition may be used for the manufacture of antimicrobial preparations and solutions. Specifically, the present composition may be used for the preparation of therapeutic medicaments which may be used to treat microbial infections, particularly fungal infections. These medicaments may be prepared as suspensions, solutions, emulsions, ointments, creams and salves for topical application on affected areas of the skin. the medicaments may also be formulated as tablets, pills, capsules or caplets which may be administered orally to a subject in need.

The antimicrobial preparations and solutions comprising the composition as disclosed herein may also be used for non-therapeutic applications. The broad spectrum of activity of the composition against bacteria and fungi are useful for the disinfection and decontamination of surfaces. As such, antimicrobial solutions comprising the composition may be added to sanitizers and sterilizing solutions which may be used in clinical or domestic settings. The solutions may also be applied to absorbent materials which may be used as antimicrobial wipes and clothes. The composition may also be added to composites and textiles to impart antimicrobial properties on the materials.

1. A composition comprising:
an oligomer of Formula (I) 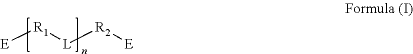
wherein R₁ is in each instance, same or different, and is independently selected from the group consisting of: 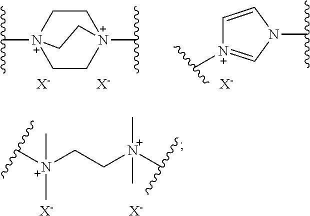
R₂ is independently selected from the group consisting of 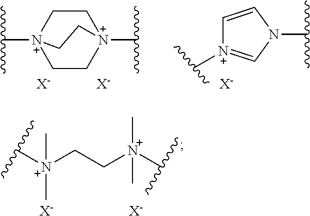
wherein X, in each instance, is same or different, and is a halogen;
L is, in each instance, independently selected from the group consisting of: optionally substituted alkyl, optionally substituted alkenyl, optionally substituted alkynyl, optionally substituted aryl, optionally substituted arylalkyl, optionally substituted arylalkenyl, optionally substituted arylalkynyl, optionally substituted alkylaryl, optionally substituted alkenylaryl, and optionally substituted alkynylaryl;
E consists of between 2 to 20 carbon atoms and is independently selected from the group consisting of: optionally substituted alkyl, optionally substituted aryl, optionally substituted arylalkyl, and optionally substituted alkylaryl;
n is an integer of between 1 to 10; and
an anti-fungal agent comprising at least one triazole group.

2. The composition according to claim 1, wherein at least one R₁ is

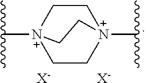

3. The composition according to claim 1, wherein R₂ is

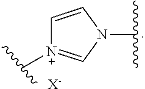

4. The composition according to claim 1, wherein E consists of between 6 to 12 carbon atoms and is independently, optionally substituted alkyl or optionally substituted aryl groups.

5. The composition according to claim 1, wherein E is a n-octyl group.

6. The composition according to claim 1, wherein L is in each instance independently selected from the group consisting of optionally substituted alkenyl, optionally substituted aryl, optionally substituted arylalkyl and optionally substituted arylalkenyl; and consists of between 2 to 20 carbon atoms.

7. The composition according to claim 1, wherein L is in each instance, independently selected from the group consisting of para-xylenyl, ortho-xylenyl and trans-2-butenyl.

8. The composition according to claim 1, wherein n is an integer from 1 to 5.

9. The composition according to claim 8, wherein n is 3.

10. The composition according to claim 1, wherein the anti-fungal agent is selected from fluconazole, itraconazole, voriconazole or combinations thereof.

11. The composition according to claim 10, wherein the anti-fungal agent is fluconazole.

12. The composition according to claim 1, wherein the weight ratio of the anti-fungal agent to the oligomer is from 1:1 to 1:1500.

13. The composition according to claim 12, wherein the oligomer is selected from the group consisting of:

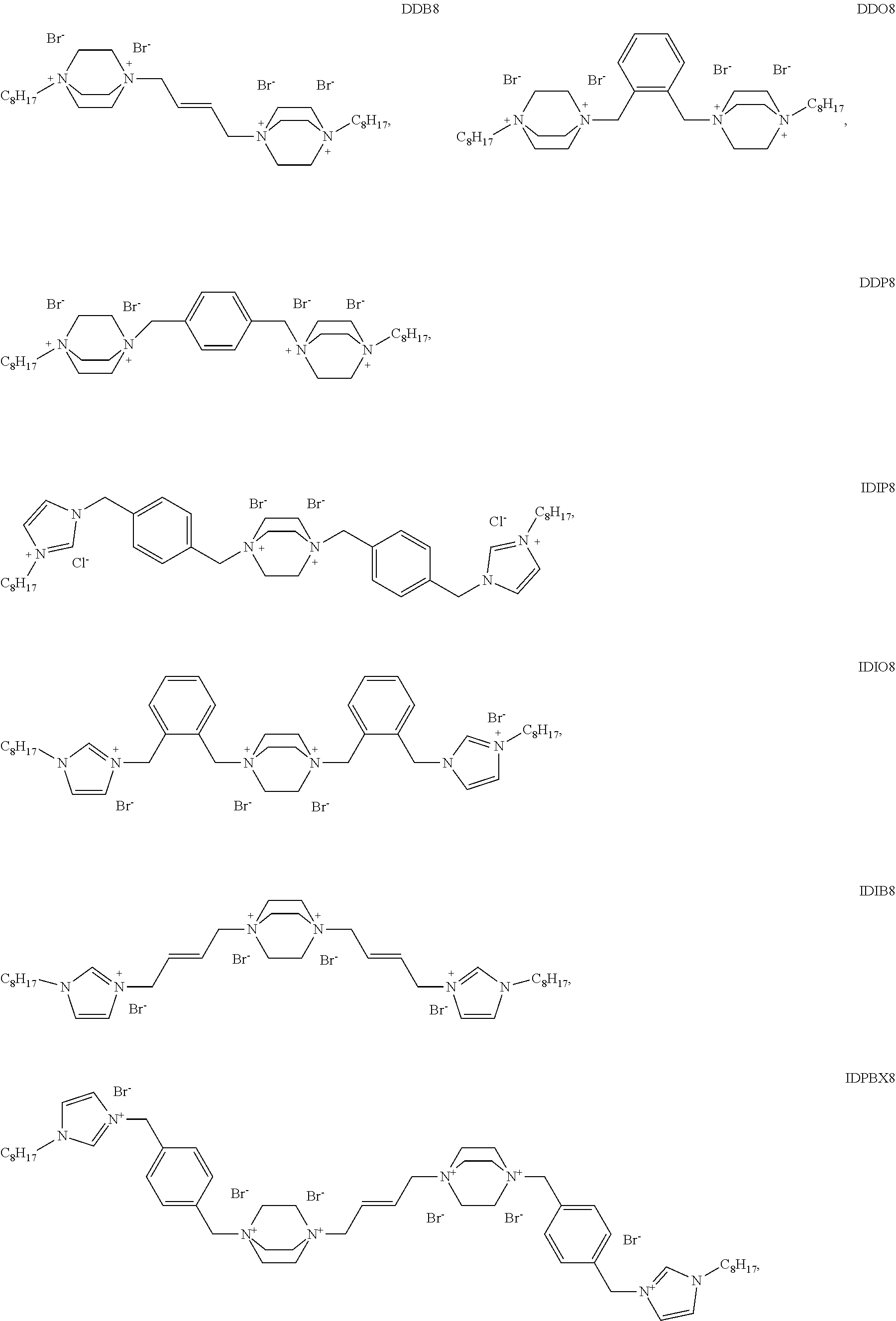

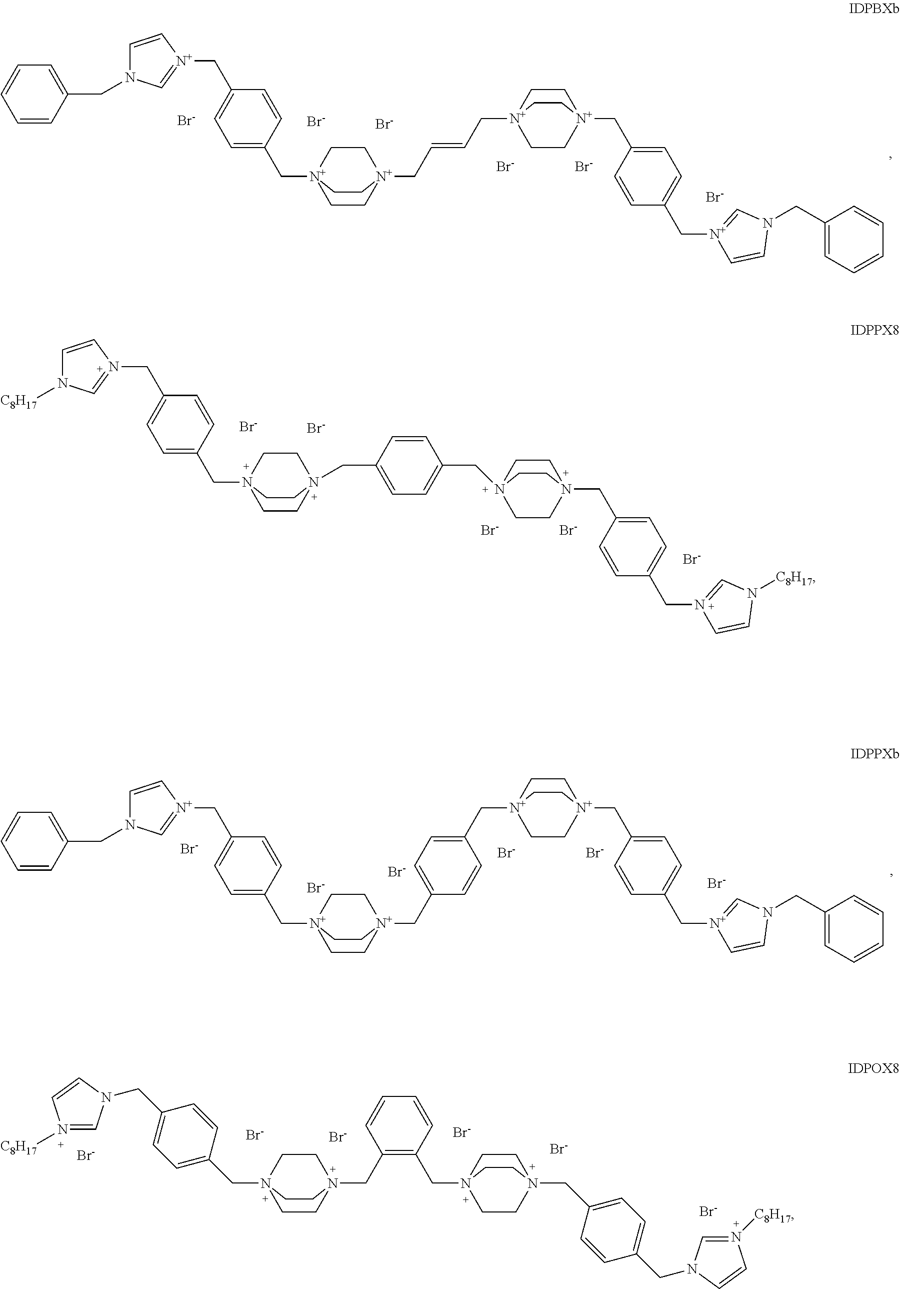

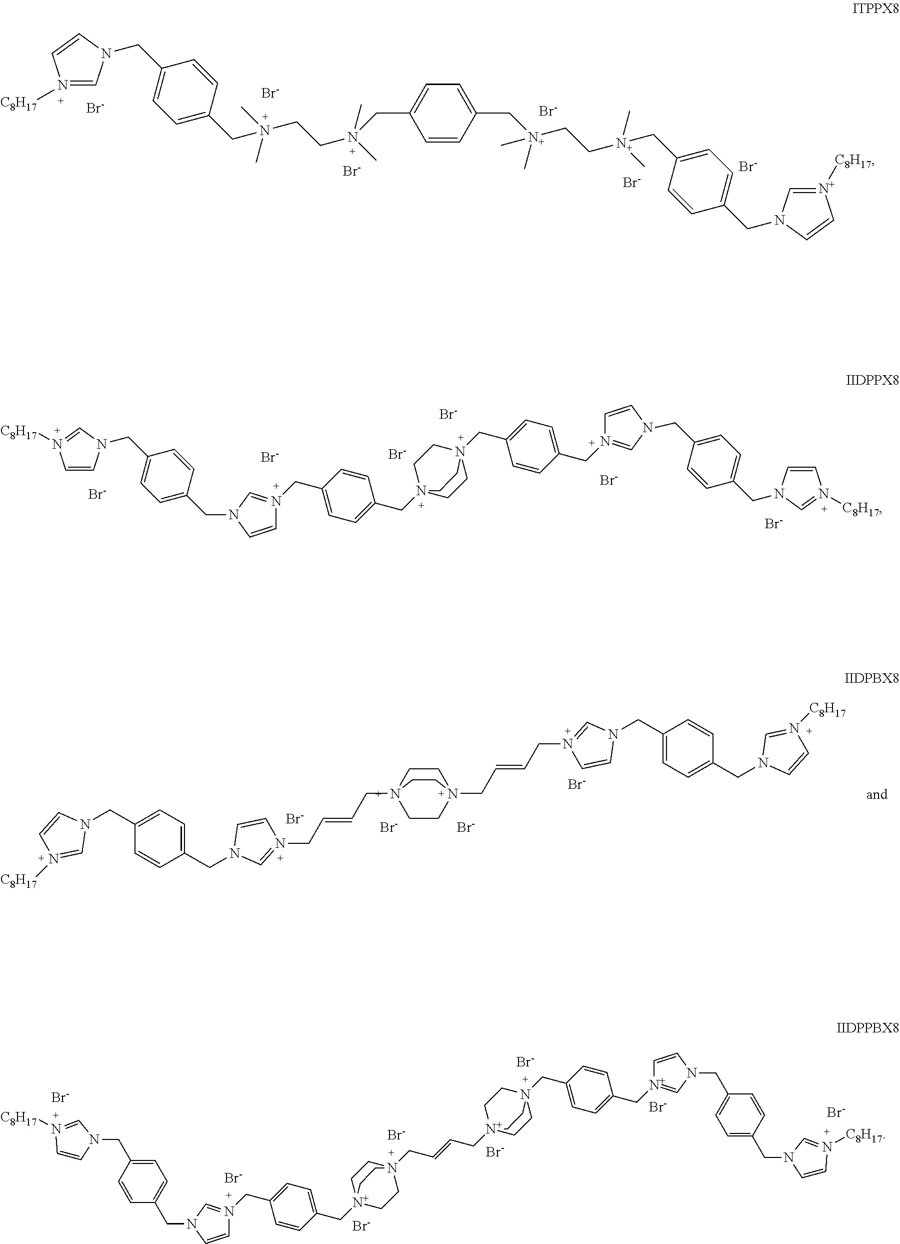

14. A method of treating microbial infections by administering the composition according to claim 1 to a subject in need thereof.

15. The method according to claim 14, wherein the microbial infection is a fungal infection caused by a Candida fungi.

16. The method according to claim 15, wherein the fungi is Candida albicans.

17. The method according to claim 14, wherein the composition is administered at a concentration of 0.2 μg/ml-150 μg/ml.

18. The method according to claim 14, wherein the composition is administered at a concentration of 1.0-4.0 μg/ml.

19. A method for killing or inhibiting microbial growth ex vivo, comprising a step of applying the composition according to claim 1.
